(19) United States
(12) Patent Application Publication
(10) Pub. No.: US 2021/0253628 A1
(43) Pub. Date: Aug. 19, 2021

(54) COMPOUNDS, COMPOSITIONS, AND METHODS FOR TREATMENT OF ANDROGEN-MEDIATED DISEASE

(71) Applicants: The Regents of the University of California, Oakland, CA (US); Ohio State Innovation Foundation, Columbus, OH (US)

(72) Inventors: Allen C. Gao, Davis, CA (US); Pui-Kai Li, Columbus, OH (US)

(73) Assignees: The Regents of the University of California, Oakland, CA (US); Ohio State Innovation Foundation, Columbus, OH (US)

(21) Appl. No.: 17/244,726

(22) Filed: Apr. 29, 2021

Related U.S. Application Data
(63) Continuation of application No. PCT/US2019/059491, filed on Nov. 1, 2019.
(60) Provisional application No. 62/754,487, filed on Nov. 1, 2018.

(51) Int. Cl. C07J 71/00; C07J 41/00; A61K 45/06; A61P 35/04

(52) U.S. Cl. CPC C07J 71/0094; A61P 35/04; A61K 45/06; C07J 41/0066

(57) ABSTRACT

Provided herein are steroid sulfatase inhibitor compounds and androgen receptor inhibitor compounds that can be useful in, for example, the treatment of cancers such as prostate cancer and breast cancer. Pharmaceutical compositions and kits including the compounds are described, as well as methods for the treatment of cancer such as prostate cancer and breast cancer.

### CROSS-REFERENCES TO RELATED APPLICATIONS

The present application is a continuation of International Pat. Appl. No. PCT/US2019/059491, filed Nov. 1, 2019, which claims priority to U.S. Provisional Pat. Appl. No. 62/754,487, filed Nov. 1, 2018, which applications are incorporated herein by reference in their entirety for all purposes.

### BACKGROUND OF THE INVENTION

Prostate cancer is the second leading cause of cancer-related deaths and the most commonly diagnosed cancer in men with an estimated 220,800 new cases yearly in the United States [Ferlay, et al., Eur J Cancer, 2013. 49(6): 1374-403; Siegel, et al., Cancer statistics, 2015. CA Cancer J Clin, 2015. 65(1): 5-29], First line treatments for prostate cancer aim to reduce circulating androgen levels through the use of androgen deprivation therapies (ADT). This is accomplished using one of two methods: surgical bilateral orchiectomy which inhibits androgen synthesis by the testes or through the use of castration-inducing drugs to reduce androgen levels and androgen receptor (AR) activation. While ADT is initially effective at reducing prostate cancer growth, after 2-3 years of treatment the majority of patients will progress to castration resistant prostate cancer (CRPC) and tumor growth will proceed even in the presence of castrate levels of androgen. At this point of disease progression, the number of therapeutic options is currently limited but is the focus of intense research to improve the outcome for patients [Harris, et al., Nat Clin Pract Urol, 2009. 6(2): 76-85].

Clinically, CRPC is defined as progression of prostate cancer in the presence of castrate levels of circulating testosterone [Cookson, et al., J Urol, 2013. 190(2): 429-38; Saad, et al., Can Urol Assoc J, 2010. 4(6): p. 380-4], Often times, the AR is either overexpressed, hyper-activated, or both leading to the transcription of downstream target genes which ultimately promotes tumor progression despite the patient having negligible levels of androgen present. The mechanisms which lead to the development of CRPC from hormone-sensitive prostate cancer are widely studied. The identified mechanisms, including AR amplification and mutation, AR co-activator and co-repressor modifications, aberrant activation and/or post-translational modification, AR splice variants, and altered steroidogenesis, each result in an increase in AR activation and signaling. This can be due to an increased amount of androgen, enhanced response to existing androgen, and activation of the AR by non-classical ligands or no ligand at all among other methods [see, Dehm, et al., Cancer Res, 2008. 68(13): 5469-77; Chang, et al., Br J Cancer, 2014. 111(7): 1249-54; Chang, et al., Proc Natl Acad Sci USA, 2011. 108(33): 13728-33; Shtivelman, et al., Oncotarget, 2014. 5(17): 7217-59; Steketee, et al., Int J Cancer, 2002. 100(3): p. 309-17].

Treatment of CRPC is currently achieved with the administration of taxanes, such as docetaxel and cabazitaxel, which interrupt the growth of fast-dividing cells through disruption of microtubule function, or with next-generation antiandrogen therapies including enzalutamide and abiraterone. The primary mechanism of antiandrogens is to inhibit AR activation either directly, by antagonizing the receptor, or indirectly by blocking androgen synthesis. Unfortunately, it is estimated that one third of patients given abiraterone and one fourth of patients given enzalutamide will fail to respond to initial treatment with these drugs [de Bono, et al., N Engl J Med, 2011. 364(21): 1995-2005; Scher, et al., N Engl J Med, 2012. 367(13): p. 1187-97], Furthermore, within 12-24 months of initiating treatment, even those who initially respond to the drugs will develop resistance.

Targeting androgen signaling via androgen deprivation therapy including recent approved therapies such as abiraterone and enzalutamide has been the mainstream of clinical interventions in castration resistant prostate cancer (CRPC). Despite these advances that provide temporary respite, there is still no cure for CRPC. Development of resistance to enzalutamide/abiraterone is eventually inevitable, with the development of several potential pathways of resistance [Scher, et al., Lancet, 2010. 375(9724): 1437-46; Kim, et al., Curr Treat Options Oncol, 2012. 13(2): 189-200.]. Recent studies have linked AR alternative splicing, particularly AR-V7, to the development of enzalutamide/abiraterone resistance [see, Nadiminty, et al., Mol Cancer Ther, 2013. 12: 1629-1637; Joseph, et al., Cancer Discov, 2013. 3(9): 1020-9; Korpal, et al., Cancer Discov, 2013. 3(9): 1030-43; Nyquist, et al., Proc Natl Acad Sci USA, 2013. 110(43): 17492-7], Previous studies demonstrate that uncontrolled intraprostatic androgen synthesis and elevated levels of cholesterol and its subsequent product dehydroepiandrosterone (DHEA) are observed in enzalutamide resistance cells [Liu, et al., Cancer Res, 2015. 75(7): 1413-1422], DHEA synthesized from cholesterol is sulfonated by DHEA sulfotransferase to DHEAS. The conversion of DHEAS to biologically active DHEA is mediated by steroid sulfatase (STS) [Purohit, et al., 212(2): 99-110], Once unconjugated, DHEA is further metabolized to the active androgens that bind the androgen receptor (AR) leading to cell proliferation.

### BRIEF SUMMARY OF THE INVENTION

Provided herein are compounds according to Formula I:

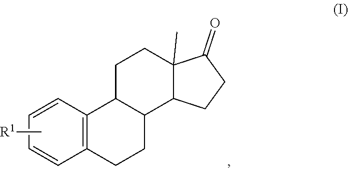

and pharmaceutically acceptable salts thereof, wherein
R¹ is —X(SO₂)Y—;
X is O and Y is NH, or X is NH and Y is O; and
R¹ is combined with two carbons of the phenyl group to which it is attached to form an oxathiazolidine dioxide.

Also provided herein are compounds according to Formula III

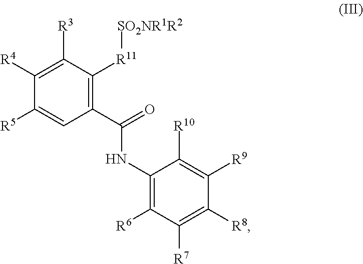

and pharmaceutically acceptable salts thereof, wherein
R¹ and R² are each independently hydrogen or C₁₋₆ alkyl;
R³, R⁴, and R⁵ are each independently hydrogen, halogen, —OH, C₁₋₆ alkyl, or C₁₋₆ alkoxy;
R⁶, R⁷, R⁸, R⁹, and R¹⁰ are each independently hydrogen, halogen, —OH, —NH₃, —NO₂, —CN, C₁₋₆haloalkyl, C₁₋₆ alkyl, C₂₋₆ alkenyl, C₂₋₆ alkynyl, or C₁₋₆ alkoxy;
R¹¹ is a bond, C₁₋₆ alkylene, NR¹², or O; and
R¹² is hydrogen or C₁₋₆ alkyl.

Also provided herein are pharmaceutical compositions comprising an antiandrogen drug and a compound according to Formula II:

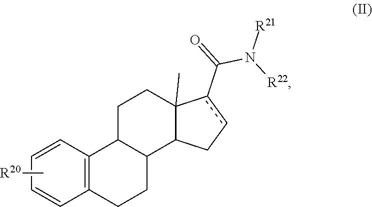

or a pharmaceutically acceptable salt thereof, wherein: ‘ ’
the dashed line represents a single bond or a double bond;
R²⁰ is —O(SO₂)NR²³R²⁴—, which is combined with two carbons of the phenyl group to which it is attached to form a 4- to 10-membered heterocycle, or

R²⁰ is —O(SO₂)NR²³R²⁵;

R²¹, R²², R²³, and R²⁵ are each independently hydrogen or C₁₋₆ alkyl; and

R²⁴ is a bond, C₁₋₆ alkylene, or C₁₋₆ alkenylene.

Steroid sulfatase inhibitors (STSi's, e.g., compounds and according to Formula I and/or Formula II) and androgen receptor inhibitors (e.g., compounds according to Formula II) can be used to enhance the therapeutic benefit of antiandrogens (e.g., bicalutamide, enzalutamide, abiraterone, darolutamide, and the like) and chemotherapeutics such as docetaxel. Also provided herein are methods for treating conditions such as cancer (e.g., prostate cancer or breast cancer). The methods include administering a compound of Formula I, Formula II, or Formula III to a subject in need thereof. In some embodiments, the methods also include administration of an antiandrogen.

### BRIEF DESCRIPTION OF THE DRAWINGS

FIG. 1A shows the structures of STSi's Si-1 to Si-5.

FIG. 1B shows the structures of STSi's Si-6 to Si-10.

FIG. 2 shows that STSi's inhibit STS activity. VCaP cells were treated with increasing doses of either Si-1 (left panel) or Si-2 (right panel) and STS activity was measured by microtiter plate cellular assay using fluorescence readout.

FIG. 3 shows the characterization of STSi's (Si-1 to Si-10) on STS enzymatic activity in VCaP cells. VCaP cells were treated with 5 μM of different STSi, and STS activity was measured by microtiter plate cellular assay using fluorescence readout.

FIG. 4 shows that STSi's inhibit prostate cancer cell growth. LNCaP, C4-2B, CWR22rv1 (rv1), DU145, PC3, and VCaP Prostate cancer cells were treated with increasing doses of either Si-1 (left panel) or Si-2 (right panel) for 48 hrs and the cell number were counted.

FIG. 5 shows the growth effects of STSi's on C4-2B cells. C4-2B prostate cancer cells were treated with increasing doses of different STSi for 48 hrs and the cell number were counted.

FIG. 6 shows that STSi's enhance enzalutamide treatment. Enzalutamide resistant C4-2BMDVR cells (left panels) and CWR22rv1 cells (right panels) were treated Si-1 (top panels) or Si-2 (bottom panels) either alone or with enzalutamide and cell number was counted.

FIG. 7 shows that STSi's enhance abiraterone treatment. Abiraterone resistant C4-2BAbiR cells (left panels) and CWR22rv1 cells (right panels) were treated Si-1 (top panels) or Si-2 (bottom panels) either alone or with abiraterone and cell number was counted.

FIG. 8A shows tumor volume in mice after VCaP xenografting, castration and tumor relapse, plotted over a 3-week course of treatment with vehicle control, Si-1 (25 mg/Kg i·p) or Si-2 (25 mg/Kg i·p).

FIG. 8B shows images of tumors collected from the treatment groups after 3 weeks.

FIG. 8C shows a plot of tumor weight collected after 3 weeks of treatment.

FIG. 8D shows a plot of body weight across treatment groups, monitored twice weekly.

FIG. 8E shows the PSA level in mouse serum, collected after 3 weeks of treatment, determined for each treatment group ELISA assay.

FIG. 8F shows IHC staining for Ki67, AR and H/E in each group. Numerical data obtained from the micrographs at left are shown at right. * p<0.05. Taken together FIGS. 8A-8E demonstrate that STSi's inhibit resistant VCaP tumor growth.

FIG. 9A. shows total cell numbers determined in VCaP cell culture, treated with 10 μM or 25 μM Si-1 or Si-2 with or without 20 μM enzalutamide for 3 days.

FIG. 9B shows the quantitation of luciferase expression in VCaP cells transiently transfected with control siRNA, STS siRNA, and PSA luciferase plasmid, following treated with 10 μM enzalutamide for 24 hours.

FIG. 9C. shows a Western blot analysis of VCaP cell lysates, prepared from cells treated with 25 μM Si-1 or Si-2 with or without 20 μM enzalutamide for 3 days.

FIG. 9D shows tumor volume in mice after VCaP xenografts, castration, and tumor relapse, plotted over a 3-week course of treatment with vehicle control, enzalutamide (25 mg/Kg p·o), Si-1 (25 mg/Kg i·p) or their combination. Tumor volumes were determined twice weekly.

FIG. 9E shows a plot of tumor weight collected from the treatment groups after 3 weeks.

FIG. 9F shows IHC staining for Ki67 and H/E staining in each group. Numerical data obtained from micrographs at left are shown at right. * p<0.05. Taken together, FIGS. 9E-9F demonstrate that STSi's improve enzalutamide treatment in vitro and in vivo.

FIG. 10 shows that STSi's inhibit breast cancer cell growth. MCF-7, MDA-MB-468, and MDA-MB-231 breast cancer cells were treated with increasing doses of either Si-1 (left panel) or Si-2 for 48 hrs (right panel), and the cell numbers were counted.

FIG. 11 shows the structure of niclosamide-sulfamate.

FIG. 12A shows total cell number in CWR22Rv1 culture treated with 0.5 μM niclosamide sulfamate (Nic-S) with or without 20 μM enzalutamide (ENZA) or 5 μM abiraterone acetate (AA), determined at 3 and 5 days.

FIG. 12B shows a Western blot analysis of C4-2B MDVR cell lysates prepared after treatment with different concentrations of Nic-S.

FIG. 12C shows a plot of tumor volume in mice bearing CWR22-rv1 tumors, over the course of combination treatment with Nic-S and enzalutamide.

FIG. 12D shows a blot of body weight measured for mice bearing CWR22-rv1 tumors, treated with enzalutamide, abiraterone acetate, or a combination thereof. *p<0.05. Taken together, FIGS. 12A-12D demonstrate that Nic-S synergistically enhances Enza/AA treatment and suppresses Wnt5A expression.

### DETAILED DESCRIPTION OF THE INVENTION

The present invention is based, in part, on the development of new compounds that can be used as steroid sulfatase inhibitors and androgen receptor inhibitors in the treatment of cancers including, without limitation, prostate cancer and breast cancer. It has also been discovered that these compounds are surprisingly effective when used in conjunction with antiandrogen drugs such as enzalutamide. The therapeutic agents and methods provided herein have been found to be effective in treating castration resistant prostate cancer and improving the efficacy of enzalutamide treatment.

### I. Definitions

As used herein, the term “alkyl,” by itself or as part of another substituent, refers to a straight or branched, saturated, aliphatic radical having the number of carbon atoms indicated. Alkyl can include any number of carbons, such as C₁₋₂, C₁₋₃, C₁₋₄, C₁₋₅, C₁₋₆, C₁₋₇, C₁₋₈, C₁₋₉, C₁₋₁₀, C₂₋₃, C₂₋₄, C₂₋₅, C₂₋₆, C₃₋₄, C₃₋₅, C₃₋₆, C₄₋₅, C₄₋₆, and C₅₋₆. For example, C₁₋₆ alkyl includes, but is not limited to, methyl, ethyl, propyl, isopropyl, butyl, isobutyl, sec-butyl, tert-butyl, pentyl, isopentyl, hexyl, etc. Alkyl can also refer to alkyl groups having up to 20 carbons atoms, such as, but not limited to heptyl, octyl, nonyl, decyl, etc. Alkyl groups can be substituted or unsubstituted. Unless otherwise specified, “substituted alkyl” groups may be substituted with one or more groups selected from halo, hydroxy, amino, alkylamino, amido, acyl, nitro, cyano, and alkoxy.

As used herein, the term “alkoxy,” by itself or as part of another substituent, refers to a group having the formula —OR, wherein R is alkyl as described above.

As used herein, the term “alkylene” refers to an alkyl group, as defined above, linking at least two other groups (i.e., a divalent alkyl radical such as “methylene” having the structure —CH₂—). The two moieties linked to the alkylene group can be linked to the same carbon atom or different carbon atoms of the alkylene group.

As used herein, the term “alkenyl,” by itself or as part of another substituent, refers to a straight chain or branched hydrocarbon having at least 2 carbon atoms and at least one double bond. Alkenyl can include any number of carbons, such as C₂, C₂₋₃, C₂₋₄, C₂₋₅, C₂₋₆, C₂₋₇, C₂₋₈, C₂₋₉, C₂₋₁₀, C₃, C₃₋₄, C₃₋₅, C₃₋₆, C₄, C₄₋₅, C₄₋₆, C₅, C₅₋₆, and C₆. Alkenyl groups can have any suitable number of double bonds, including, but not limited to, 1, 2, 3, 4, 5 or more. Examples of alkenyl groups include, but are not limited to, vinyl (ethenyl), propenyl, isopropenyl, 1-butenyl, 2-butenyl, isobutenyl, butadienyl, 1-pentenyl, 2-pentenyl, isopentenyl, 1,3-pentadienyl, 1,4-pentadienyl, 1-hexenyl, 2-hexenyl, 3-hexenyl, 1.3-hexadienyl, 1,4-hexadienyl, 1,5-hexadienyl, 2,4-hexadienyl, or 1,3,5-hexatrienyl. Alkenyl groups can be substituted or unsubstituted. Unless otherwise specified, “substituted alkenyl” groups may be substituted with one or more moieties selected from halo, hydroxy, amino, alkylamino, alkoxy, haloalkyl, carboxy, amido, nitro, oxo, and cyano.

As used herein, the term “alkenylene” refers to an alkenyl group, as defined above, linking at least two other groups (i.e., a divalent alkenyl radical such as “methine” having the structure —CH═). The two moieties linked to the alkenylene group can be linked to the same carbon atom or different carbon atoms of the alkenylene group.

As used herein, the term “alkynyl,” by itself or as part of another substituent, refers to either a straight chain or branched hydrocarbon having at least 2 carbon atoms and at least one triple bond. Alkynyl can include any number of carbons, such as C₂, C₂₋₃, C₂₋₄, C₂₋₅, C₂₋₆, C₂₋₇, C₂₋₈, C₂₋₉, C₂₋₁₀, C₃, C₃₋₄, C₃₋₅, C₃₋₆, C₄, C₄₋₅, C₄₋₆, C₅, C₅₋₆, and C₆. Examples of alkynyl groups include, but are not limited to, acetylenyl, propynyl, 1-butynyl, 2-butynyl, isobutynyl, sec-butynyl, butadiynyl, 1-pentynyl, 2-pentynyl, isopentynyl, 1,3-pentadiynyl, 1.4-pentadiynyl, 1-hexynyl, 2-hexynyl, 3-hexynyl, 1,3-hexadiynyl, 1,4-hexadiynyl, 1.5-hexadiynyl, 2,4-hexadiynyl, or 1,3,5-hexatriynyl. Alkynyl groups can be substituted or unsubstituted. Unless otherwise specified, “substituted alkynyl” groups may be substituted with one or more moieties selected from halo, hydroxy, amino, alkylamino, alkoxy, haloalkyl, carboxy, amido, nitro, oxo, and cyano.

As used herein, the terms “halo” and “halogen” refer to fluorine, chlorine, bromine and iodine.

As used herein, the term “haloalkyl,” by itself or as part of another substituent, refers to an alkyl group where some or all of the hydrogen atoms are replaced with halogen atoms. As for alkyl groups, haloalkyl groups can have any suitable number of carbon atoms, such as C₁₋₆. For example, haloalkyl includes trifluoromethyl, fluoromethyl, etc. In some instances, the term “perfluoro” can be used to define a compound or radical where all the hydrogens are replaced with fluorine. For example, perfluoromethyl refers to 1,1,1-trifluoromethyl.

As used herein the term “heterocyclyl,” by itself or as part of another substituent, refers to a saturated ring system having from 3 to 12 ring members and from 1 to 4 heteroatoms of N, O and S. Additional heteroatoms can also be useful, including, but not limited to, B, Al, Si and P. The heteroatoms can be oxidized to form moieties such as, but not limited to, —S(O)— and —S(O)₂-. Heterocyclyl groups can include any number of ring atoms, such as, C₃₋₆, C₄₋₆, C₅₋₆, C₃₋₈, C₄₋₈, C₅₋₈, C₆₋₈, C₃₋₉, C₃₋₁₀, C₃₋₁₁, or C₃₋₁₂, wherein at least one of the carbon atoms is replaced by a heteroatom. Any suitable number of carbon ring atoms can be replaced with heteroatoms in the heterocyclyl groups, such as 1, 2, 3, or 4, or 1 to 2, 1 to 3, 1 to 4, 2 to 3, 2 to 4, or 3 to 4. The heterocyclyl group can include groups such as aziridine, azetidine, pyrrolidine, piperidine, azepane, azocane, quinuclidine, pyrazolidine, imidazolidine, piperazine (1,2-, 1,3- and 1,4-isomers), oxirane, oxetane, tetrahydrofuran, oxane (tetrahydropyran), oxepane, thiirane, thietane, thiolane (tetrahydrothiophene), thiane (tetrahydrothiopyran), oxazolidine, isoxazolidine, thiazolidine, isothiazolidine, dioxolane, dithiolane, morpholine, thiomorpholine, dioxane, or dithiane. The heterocyclyl groups can also be fused to aromatic or non-aromatic ring systems to form members including, but not limited to, indoline. Heterocyclyl groups can be unsubstituted or substituted. Unless otherwise specified, “substituted heterocyclyl” groups can be substituted with one or more groups selected from halo, hydroxy, amino, oxo (═O), alkylamino, amido, acyl, nitro, cyano, and alkoxy.

The heterocyclyl groups can be linked via any position on the ring. For example, aziridine can be 1- or 2-aziridine, azetidine can be 1- or 2-azetidine, pyrrolidine can be 1-, 2- or 3-pyrrolidine, piperidine can be 1-, 2-, 3- or 4-piperidine, pyrazolidine can be 1-, 2-, 3-, or 4-pyrazolidine, imidazolidine can be 1-, 2-, 3- or 4-imidazolidine, piperazine can be 1-, 2-, 3- or 4-piperazine, tetrahydrofuran can be 1- or 2-tetrahydrofuran, oxazolidine can be 2-, 3-, 4- or 5-oxazolidine, isoxazolidine can be 2-, 3-, 4- or 5-isoxazolidine, thiazolidine can be 2-, 3-, 4- or 5-thiazolidine, isothiazolidine can be 2-, 3-, 4- or 5-isothiazolidine, and morpholine can be 2-, 3- or 4-morpholine.

When heterocyclyl includes 3 to 8 ring members and 1 to 3 heteroatoms, representative members include, but are not limited to, pyrrolidine, piperidine, tetrahydrofuran, oxane, tetrahydrothiophene, thiane, pyrazolidine, imidazolidine, piperazine, oxazolidine, isoxazolidine, thiazolidine, isothiazolidine, morpholine, thiomorpholine, dioxane and dithiane. Heterocyclyl can also form a ring having 5 to 6 ring members and 1 to 2 heteroatoms, with representative members including, but not limited to, pyrrolidine, piperidine, tetrahydrofuran, tetrahydrothiophene, pyrazolidine, imidazolidine, piperazine, oxazolidine, isoxazolidine, thiazolidine, isothiazolidine, and morpholine.

As used herein, the term “hydroxy” refers to the moiety —OH.

As used herein, the term “oxo” refers to an oxygen atom that is double-bonded to a compound (i.e., O═).

As used herein, the term “amino” refers to a moiety —NR₂, wherein each R group is H or alkyl. An amino moiety can be ionized to form the corresponding ammonium cation. “Alkylamino” refers to an amino moiety wherein at least one of the R groups is alkyl.

As used herein, the term “amido” refers to a moiety —NRC(O)R or —C(O)NR₂, wherein each R group is H or alkyl.

As used herein, the term “acyl” refers to the moiety —C(O)R, wherein each R group is alkyl.

As used herein, the term “nitro” refers to the moiety —NO₂.

As used herein, the term “cyano” refers to a carbon atom triple-bonded to a nitrogen atom (i.e., the moiety —C≡N).

As used herein, the term “carboxy” refers to the moiety —C(O)OH.

As used herein, the term “oxathiazolidine dioxide” refers to a moiety having the structure:

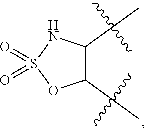 [SMILES: *C1NS(=O)(=O)OC1*]

wherein the wavy lines indicate points of attachment to other atoms in the molecule containing the oxathiazolidine dioxide.

As used herein, the term “oxathiazine dioxide” refers to a moiety having the structure:

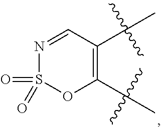 [SMILES: *C1=C(*)OS(=O)(=O)N=C1]

wherein the wavy lines indicate points of attachment to other atoms in the molecule containing the oxathiazine dioxide.

As used herein, the term “dihydro-oxathiazine dioxide” refers to a moiety having the structure:

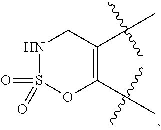 [SMILES: *C1=C(*)OS(=O)(=O)NC1]

wherein the wavy lines indicate points of attachment to other atoms in the molecule containing the dihydro-oxathiazine dioxide.

As used herein, the term “salt” refers to an acid salt or base salt of an active agent such as a steroid sulfatase inhibitor or an androgen receptor inhibitor. Acid salts of basic active agents include mineral acid salts (e.g., salts formed by using hydrochloric acid, hydrobromic acid, phosphoric acid, and the like), organic acid salts (e.g., salts formed using acetic acid, propionic acid, glutamic acid, citric acid, and the like), and quaternary ammonium salts (e.g., salts formed via reaction of an amine with methyl iodide, ethyl iodide, or the like). It is understood that the pharmaceutically acceptable salts are non-toxic.

Acidic active agents may be contacted with bases to provide base salts such as alkali and alkaline earth metal salts, such as sodium, lithium, potassium, calcium, magnesium, as well as ammonium salts, such as ammonium, trimethyl-ammonium, diethylammonium, and tris-(hydroxymethyl)-methyl-ammonium salts.

The neutral forms of the active agents can be regenerated by contacting the salt with a base or acid and isolating the parent compound in the conventional manner if desired. In some embodiments, the parent form of the compound may differ from various salt forms in certain physical properties, such as solubility in polar solvents, but otherwise the salts forms may be equivalent to the parent form of the compound.

By “pharmaceutically acceptable,” it is meant that the excipient is compatible with the other ingredients of the formulation and is not deleterious to the recipient thereof. As used herein, the term “pharmaceutically acceptable excipient” refers to a substance that aids the administration of an active agent to a subject. Useful pharmaceutical excipients include, but are not limited to, binders, fillers, disintegrants, lubricants, glidants, coatings, sweeteners, flavors and colors.

As used herein, the terms “effective amount” and “therapeutically effective amount” refer to a dose of a compound such a steroid sulfatase inhibitor, an androgen receptor inhibitor, or an antiandrogen that produces therapeutic effects for which it is administered. The exact dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); Goodman & Gilman's The Pharmacological Basis of Therapeutics, 11^{th} Edition, 2006, Brunton, Ed., McGraw-Hill; and Remington; The Science and Practice of Pharmacy, 21^{st} Edition, 2005, Hendrickson, Ed., Lippincott, Williams & Wilkins).

As used herein, the term “cancer” is intended to include any member of a class of diseases characterized by the uncontrolled growth of aberrant cells. The term includes all known cancers and neoplastic conditions, whether characterized as malignant, benign, recurrent, soft tissue, or solid, and cancers of all stages and grades including advanced, recurrent, pre- and post-metastatic cancers. Additionally, the term includes androgen-independent, castrate-resistant, castration recurrent, hormone-resistant, drug-resistant, and metastatic castrate-resistant cancers. Examples of different types of cancer include, but are not limited to, prostate cancer (e.g., prostate adenocarcinoma); breast cancers (e.g., triple-negative breast cancer, ductal carcinoma in situ, invasive ductal carcinoma, tubular carcinoma, medullary carcinoma, mucinous carcinoma, papillary carcinoma, cribriform carcinoma, invasive lobular carcinoma, inflammatory breast cancer, lobular carcinoma in sins, Paget's disease, Phyliodes tumors); gynecological cancers (e.g., ovarian, cervical, uterine, vaginal, and vulvar cancers); lung cancers (e.g., non-small cell lung cancer, small cell lung cancer, mesothelioma, carcinoid tumors, lung adenocarcinoma); digestive and gastrointestinal cancers such as gastric cancer (e.g., stomach cancer), colorectal cancer, gastrointestinal stromal tumors (GIST), gastrointestinal carcinoid tumors, colon cancer, rectal cancer, anal cancer, bile duct cancer, small intestine cancer, and esophageal cancer; thyroid cancer; gallbladder cancer; liver cancer; pancreatic cancer; appendix cancer; renal cancer (e.g., renal cell carcinoma); cancer of the central nervous system (e.g., glioblastoma, neuroblastoma); skin cancer (e.g., melanoma); bone and soft tissue sarcomas (e.g., Ewing's sarcoma); lymphomas, choriocarcinoma; urinary cancers (e.g., urothelial bladder cancer); head and neck cancers; and bone marrow and blood cancers (e.g., chronic lymphocytic leukemia, lymphoma). As used herein, a “tumor” comprises one or more cancerous cells.

As used herein, the terms “antiandrogen” and “antiandrogen drug” refer to compounds that alter the androgen pathway by blocking the androgen receptors, competing for binding sites on the cell's surface, or affecting or mediating androgen production. Antiandrogens are useful for treating several diseases including, but not limited to, prostate cancer. Examples of antiandrogens include, but are not limited to, enzalutamide, abiraterone, bicalutamide, and darolutamide.

As used herein, the terms “about” and “around” indicate a close range around a numerical value when used to modify that specific value. If “X” were the value, for example, “about X” or “around X” would indicate a value from 0.9X to 1.1X, e.g., a value from 0.95X to 1.05X, or a value from 0.98X to 1.02X, or a value from 0.99X to 1.01X. Any reference to “about X” or “around X” specifically indicates at least the values X, 0.9X, 0.91X, 0.92X, 0.93X, 0.94X, 0.95X, 0.96X, 0.97X, 0.98X, 0.99X, 1.01X, 1.02X, 1.03X, 1.04X, 1.05X, 1.06X, 1.07X, 1.08X, 1.09X, and 1.1X, and values within this range.

### II. Steroid Sulfatase Inhibitors

Development of resistance to enzalutamide/abiraterone is eventually inevitable Emerging clinical evidence showed the serum level of DHEAS concentration is highly elevated in advanced prostate cancer, and may serve as an ample pool for intracrine androgen synthesis. STS is an enzyme involved in the local production of androgens and estrogens in target organs [Purohit, supra]. The present invention was developed, in part, to identify new steroid sulfatase inhibitors for blocking STS activity as a therapeutic approach to treat STS-activated hormone related cancers, including resistant prostate and breast cancers. As described in more detail below, several STS inhibitors (STSi's) were synthesized and found to inhibit STS activity in prostate cancer cells. Inhibition of STS by STSi's inhibited the growth of enzalutamide-resistant C4-2B MDVR cells, abiraterone-resistant C4-2BAbiR cells, and VCaP and CWR22Rv1 cells. STSi resensitized enzalutamide-resistant C4-2B MDVR and CWR22Rv1 cells to enzalutamide treatment. Similarly, STSi's also resensitized abiraterone resistant cells to abiraterone treatment. Furthermore, STSi's significantly inhibited tumor growth of resistant VCaP prostate tumor growth in castrated male mice. Additionally, it was demonstrated that STSi's inhibited the growth of resistant MDA-MB-231 breast cancer cells.

Accordingly, provided herein are compounds according to Formula I:

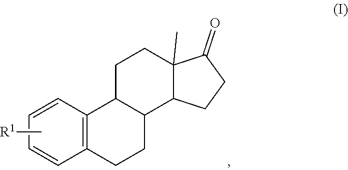

and pharmaceutically acceptable salts thereof, wherein:
R¹ is —X(SO₂)Y—;
X is O and Y is NH, or X is NH and Y is O; and
R¹ is combined with two carbons of the phenyl group to which it is attached to form an oxathiazolidine dioxide.

In some embodiments, the compound has a structure according to Formula Ia:

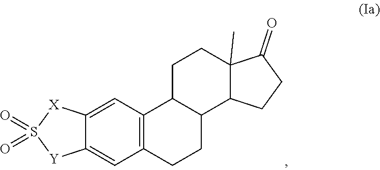

wherein one of X and Y is NH, and wherein the other of X and Y is O.

In some embodiments, the compound is:

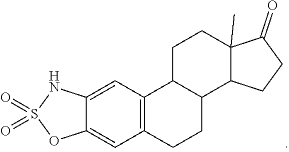 [SMILES: CC12CCC3c4cc5c(cc4CCC3C1CCC2=O)OS(=O)(=O)N5]

In some embodiments, the compound has a structure according to Formula Ib:

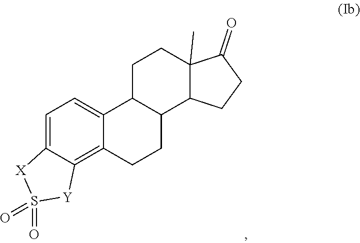

wherein one of X and Y is NH, and wherein the other of X and Y is O.

In some embodiments, the compound of Formula Ib is

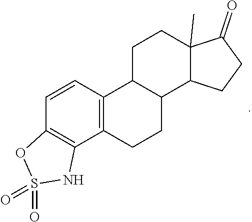 [SMILES: CC12CCC3c4ccc5c(c4CCC3C1CCC2=O)NS(=O)(=O)O5]

### III. Androgen Receptor Inhibitors

Androgen receptor (AR) variants are known to be upregulated in certain cancers such as castration-resistant prostate cancer (CRPC). Expression of AR variants is associated with prostate cancer progression and resistance to AR-targeted therapy (Mostaghel et al., Clin Cancer Res 2011; 17:5913-25; Schrader et al., Eur Urol 2013; 64:169-70; Zhang et al., PLoS One 2011; 6:e27970; Sun et al., J Clin Invest 2010; 120:2715-30). AR variant AR-V7, which is encoded by contiguous splicing of AR exons 1/2/3/CE3, is known for its prevalence in prostate cancer samples (7, 12, 16) and can induce castration resistant cell growth in vitro and in vivo (7, 17). The present inventors previously found that niclosamide (2′,5-dichloro-4′-nitrosalicylanilide) can be used as an AR inhibitor to overcome enzalutamide resistance and enhances enzalutamide therapy in prostate cancer cells.

Also provided herein are compounds according to Formula III:

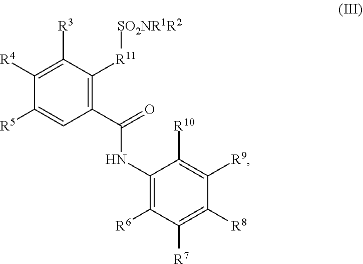

and pharmaceutically acceptable salts thereof, wherein:
R¹ and R² are each independently hydrogen or C₁₋₆ alkyl;
R³, R⁴, and R⁵ are each independently hydrogen, halogen, —OH, C₁₋₆ alkyl, or C₁₋₆ alkoxy;
R⁶, R⁷, R⁸, R⁹, and R¹⁰ are each independently hydrogen, halogen, —OH, —NH₃, —NO₂, —CN, C₁₋₆haloalkyl (e.g., —CF₃ or —CCl₃), C₁₋₆ alkyl, C₂₋₆ alkenyl, C₂₋₆ alkynyl, or C₁₋₆ alkoxy;
R¹¹ is a bond, C₁₋₆ alkylene, NR¹², or O; and
R¹² is hydrogen or C₁₋₆ alkyl.

In some embodiments, R¹¹ is C₁₋₆ alkylene (e.g., methylene, ethylene, or n-propylene). In some embodiments, R¹¹ is NR¹² or O. In some embodiments, R¹¹ is NR¹² and R¹² is H. In some embodiments, R¹¹ is NR¹² and R¹² is C₁₋₆ alkyl (e.g., methyl, ethyl, 72-propyl, isopropyl, n-butyl, tert-butyl, sec-butyl, n-pentyl, branched pentyl, n-hexyl, or branched hexyl).

In some embodiments, R¹¹ is a bond. In some embodiments, the compound has a structure according to Formula IIIa:

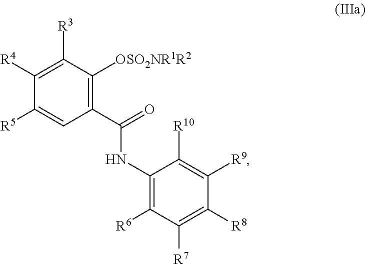

wherein, R¹ and R² are each independently hydrogen or C₁₋₆ alkyl; R³, R⁴, and R⁵ are each independently hydrogen, halogen, —OH, C₁₋₆ alkyl, or C₁₋₆ alkoxy; and R⁶, R⁷, R⁸, R⁹, and R¹⁰ are each independently hydrogen, halogen, —OH, —NH₃, —NO₂, —CN, C₁₋₆ haloalkyl, C₁₋₆ alkyl, C₂₋₆ alkenyl, C₂₋₆ alkynyl, or C₁₋₆ alkoxy.

In some embodiments, R¹ and R² is H. In some embodiments, R¹ is H and R² is C₁₋₆ alkyl (e.g., methyl, ethyl, n-propyl, isopropyl, n-butyl, tert-butyl, sec-butyl, n-pentyl, branched pentyl, n-hexyl, or branched hexyl). In some embodiments R¹ and R² are C₁₋₆ alkyl.

In some embodiments, R³, R⁴, R⁵, R⁶, R⁷, R⁸, R⁹, and R¹⁰ are each independently hydrogen, halogen, or —NO₂. In some embodiments, R⁵ is halogen (e.g., fluoro, chloro, or bromo) and R³, R⁴, R⁶, R⁷, R⁸, R⁹, and R¹⁰ are each independently hydrogen, halogen, or —NO₂. In some embodiments, R⁵ is halogen (e.g., fluoro, chloro, or bromo) and R³ and R⁴ are each independently hydrogen or halogen. In some such embodiments, R⁸ is —NO₂.

In some embodiments, R⁸ is —NO₂ and R³, R⁴, R⁵, R⁶, R⁷, R⁹, and R¹⁰ are each independently hydrogen or halogen. In some embodiments, R⁸ is halogen (e.g., fluoro, chloro, or bromo) and R⁶, R⁷, R⁹, and R¹⁰ are each independently hydrogen or halogen. In some such embodiments, R⁸ is —NO₂, R⁶ is halogen, and R⁷, R⁹, and R¹⁰ are H. In some such embodiments, R⁵ is halogen and R³ and R⁴ are each independently hydrogen or halogen.

In some embodiments, R¹ and R² are hydrogen in compounds of Formula III or IIIa; and R³, R⁴, R⁵, R⁶, R⁷, R⁸, R⁹, and R¹⁰ are each independently hydrogen, halogen, or —NO₂.

In some embodiments, compounds of Formula III or IIIa are provided wherein:
one of R³, R⁴, and R⁵ is halogen and two of R³, R⁴, and R⁵ are hydrogen; and
one of R⁶, R⁷, R⁸, R⁹, and R¹⁰ is halogen, one of R⁶, R⁷, R⁸, R⁹, and R¹⁰ is —NO₂, and three of R⁶, R⁷, R⁸, R⁹, and R¹⁰ are hydrogen. In some such embodiments, R¹ and R² are hydrogen.

In some embodiments, compounds of Formula III or IIIa are provided wherein:
one of R³, R⁴, and R⁵ is chloro and two of R³, R⁴, and R⁵ are hydrogen; and
one of R⁶, R⁷, R⁸, R⁹, and R¹⁰ is chloro, one of R⁶, R⁷, R⁸, R⁹, and R¹⁰ is —NO₂, and three of R⁶, R⁷, R⁸, R⁹, and R¹⁰ are hydrogen. In some such embodiments, R¹ and R² are hydrogen.

In some embodiments, the compound of Formula III or IIIa is:

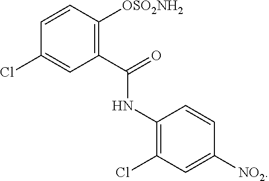

### IV. Antiandrogen Compositions and Other Pharmaceutical Compositions

Advantageously, compounds according to the present disclosure can be used in conjunction with antiandrogen drugs for the synergistic treatment of diseases such as cancer. Use of the compounds can result, for example, in the re-sensitization of antiandrogen-resistant cancer (e.g., antiandrogen-resistant prostate cancer or breast cancer) to antiandrogen therapy. Combination therapies according to the present disclosure may employ steroidal antiandrogens (e.g., cyproterone acetate, abiraterone, and the like) and/or non-steroidal antiandrogens (e.g., enzalutamide, flutamide, nilutamide, and bicalutamide). These and other antiandrogens are described, for example by Schröder et al. ((2009) “Steroidal Antiandrogens.” In: Jordan V. C., Furr B. J. (eds) Hormone Therapy in Breast and Prostate Cancer. Cancer Drug Discovery and Development. Humana Press) and Kolvenbag, et al. ((2009) “Nonsteroidal Antiandrogens.” In: Jordan V. C., Furr B. J. (eds) Hormone Therapy in Breast and Prostate Cancer. Cancer Drug Discovery and Development. Humana Press).

Some embodiments of the present disclosure provide a pharmaceutical composition comprising an antiandrogen drug and a compound according to Formula II:

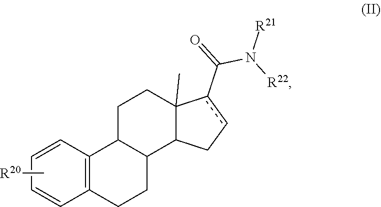

wherein:
the dashed line represents a single bond or a double bond;
R²⁰ is —O(SO₂)NR²³R²⁴—, which is combined with two carbons of the phenyl group to which it is attached to form a 4- to 10-membered heterocycle, or
R²⁰ is —O(SO₂)NR²³R²⁵;
R²¹, R²², R²³, and R²⁵ are each independently hydrogen or C₁₋₆ alkyl; and R²⁴ is a bond, C₁₋₆ alkylene, or C₁₋₆ alkenylene.

In some embodiments, the compound has a structure according to Formula IIa:

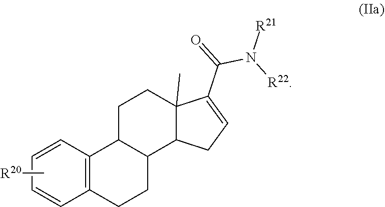

In some embodiments, R²¹ and R²² are each independently hydrogen or C₁₋₃ alkyl in compounds of Formula II or Formula IIa. In some embodiments, R²¹ is hydrogen, methyl, ethyl, propyl, isopropyl in compounds of Formula II or Formula IIa; and R²² is hydrogen, propyl, or isopropyl. In some such embodiments, R²⁰ is —O(SO₂)NR²³R²⁴—, and R²⁴ is C₁₋₆ alkylene or C₁₋₆ alkenylene. In some embodiments, R²⁰ is combined with two carbons of the phenyl group to which it is attached to form an oxathiazine dioxide or a dihydro-oxathiazine dioxide. In some embodiments, R²⁰ is —O(SO₂)—NR²³R²⁵ in compounds of Formula II and Formula IIa.

In some embodiments, R²¹ is hydrogen, methyl, ethyl, propyl, or isopropyl; and R²² is hydrogen, propyl, or isopropyl. Compounds according to Formula II and Formula IIa can be prepared as described below and, for example, in U.S. Pat. No. 6,399,595.

In some embodiments, the steroid sulfatase inhibitor compound has a structure selected from the group consisting of:

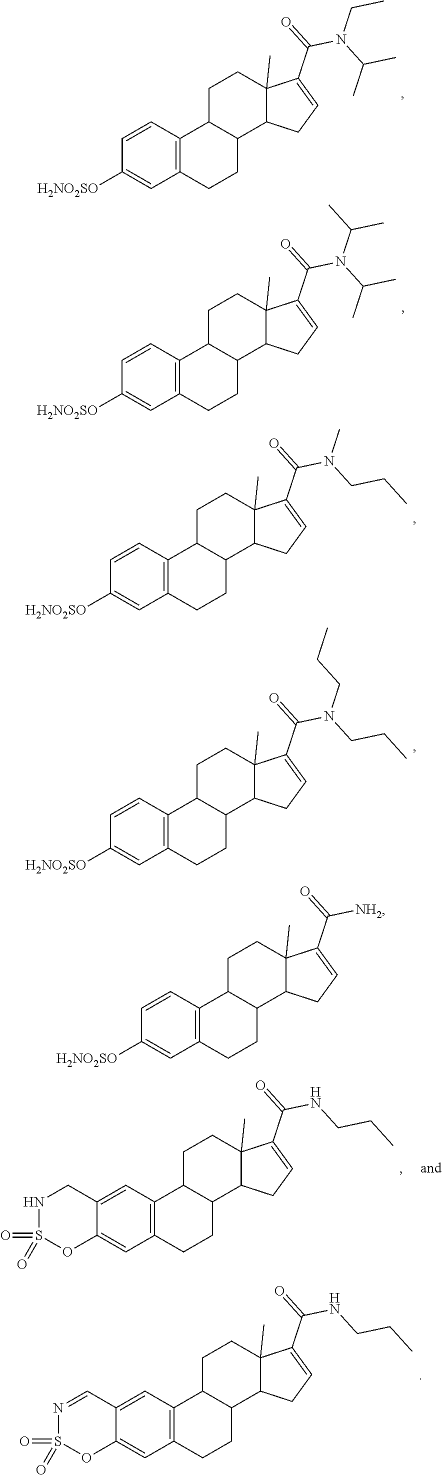

In some embodiments, the antiandrogen drug is selected from the group consisting of bicalutamide, apalutamide, enzalutamide, abiraterone, darolutamide, and a combination thereof.

Also provided herein are compositions comprising: (i) compounds of Formula I, Formula III, or a combination thereof, and (ii) an antiandrogen drug as set forth above.

Typically, pharmaceutical compositions will contain one or more pharmaceutically acceptable excipients in combination with the steroid sulfatase inhibitor and/or the androgen receptor inhibitor.

The pharmaceutical compositions are generally made by admixing an STS inhibitor (e.g., compound according to Formula I or II) and/or an AR inhibitor (e.g., a compound according to Formula III), optionally an antiandrogen drug (e.g., bicalutamide, apalutamide, enzalutamide, darolutamide, abiraterone acetate, or a combination thereof), and a pharmaceutically acceptable carrier and/or excipient or diluent. Such compositions are suitable for pharmaceutical use in a human or other animal. The pharmaceutical compositions may be prepared by any of the methods well-known in the art of pharmacy (see, e.g., Lieberman, Pharmaceutical Dosage Forms (vols. 1-3, 1992); Lloyd, The Art, Science and Technology of Pharmaceutical Compounding (1999); and Remington; The Science and Practice of Pharmacy, 21^{st} Edition, 2005, Hendrickson, Ed., Lippincott, Williams & Wilkins). Pharmaceutically acceptable carriers include any of the standard pharmaceutical carriers, buffers and excipients, including phosphate-buffered saline solution, water, and emulsions (such as an oil/water or water/oil emulsion), and various types of wetting agents and/or adjuvants. Preferred pharmaceutical carriers will depend, in part, upon the intended mode of administration of the active agent.

The pharmaceutical compositions can include a combination of drugs (e.g., compounds according to Formulas I, II, and/or III and an antiandrogen drug such as enzalutamide, abiraterone, bicalutamide, darolutamide, and/or apalutamide), or any pharmaceutically acceptable salts thereof, as active ingredients and a pharmaceutically acceptable carrier and/or excipient or diluent. A pharmaceutical composition may optionally contain other therapeutic ingredients.

The compositions (e.g., combinations of STS inhibitors, AR inhibitors, and/or antiandrogen drugs) can be combined as the active ingredients in intimate admixture with a suitable pharmaceutical carrier and/or excipient according to conventional pharmaceutical compounding techniques. Any carrier and/or excipient suitable for the form of preparation desired for administration is contemplated for use with the compounds disclosed herein.

The pharmaceutical compositions include those suitable for topical, parenteral, pulmonary, nasal, rectal, or oral administration. The most suitable route of administration in any given case will depend in part on the nature and severity of the cancer (e.g., prostate or breast cancer) condition and also optionally the stage of the cancer.

Other pharmaceutical compositions include those suitable for systemic (enteral or parenteral) administration. Systemic administration includes oral, rectal, sublingual, or sublabial administration. Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. In particular embodiments, pharmaceutical compositions may be administered intratumorally.

Compositions for pulmonary administration include, but are not limited to, dry powder compositions consisting of the powder of a compound described herein, or a salt thereof, and the powder of a suitable carrier and/or lubricant. The compositions for pulmonary administration can be inhaled from any suitable dry powder inhaler device known to a person skilled in the art.

The pharmaceutical compositions may be in a form suitable for oral use. Suitable compositions for oral administration include, but are not limited to, tablets, troches, lozenges, aqueous or oily suspensions, dispersible powders or granules, emulsions, hard or soft capsules, syrups, elixirs, solutions, buccal patches, oral gels, chewing gums, chewable tablets, effervescent powders, and effervescent tablets. Such compositions can contain one or more agents selected from sweetening agents, flavoring agents, coloring agents, antioxidants, and preserving agents in order to provide pharmaceutically elegant and palatable preparations.

Tablets generally contain the active ingredients in admixture with non-toxic pharmaceutically acceptable excipients, including: inert diluents, such as cellulose, silicon dioxide, aluminum oxide, calcium carbonate, sodium carbonate, glucose, mannitol, sorbitol, lactose, calcium phosphate, and sodium phosphate; granulating and disintegrating agents, such as corn starch and alginic acid; binding agents, such as polyvinylpyrrolidone (PVP), cellulose, polyethylene glycol (PEG), starch, gelatin, and acacia; and lubricating agents such as magnesium stearate, stearic acid, and talc. The tablets can be uncoated or coated, enterically or otherwise, by known techniques to delay disintegration and absorption in the gastrointestinal tract and thereby provide a sustained action over a longer period. For example, a time delay material such as glyceryl monostearate or glyceryl distearate can be employed. Tablets can also be coated with a semi-permeable membrane and optional polymeric osmogents according to known techniques to form osmotic pump compositions for controlled release. Compositions for oral administration can be formulated as hard gelatin capsules wherein the active ingredient is mixed with an inert solid diluent (such as calcium carbonate, calcium phosphate, or kaolin), or as soft gelatin capsules wherein the active ingredients are mixed with water or an oil medium (such as peanut oil, liquid paraffin, or olive oil).

The pharmaceutical compositions can also be in the form of an injectable aqueous or oleaginous solution or suspension. Sterile injectable preparations can be formulated using non-toxic parenterally-acceptable vehicles including water, Ringer's solution, and isotonic sodium chloride solution, and acceptable solvents such as 1,3-butane diol. In addition, sterile, fixed oils are conventionally employed as a solvent or suspending medium. For this purpose any bland fixed oil can be employed including synthetic mono- or diglycerides. In addition, fatty acids such as oleic acid find use in the preparation of injectables.

Aqueous suspensions contain the active agents in admixture with excipients suitable for the manufacture of aqueous suspensions. Such excipients include, but are not limited to: suspending agents such as sodium carboxymethylcellulose, methylcellulose, oleagino-propylmethylcellulose, sodium alginate, polyvinyl-pyrrolidone, gum tragacanth and gum acacia; dispersing or wetting agents such as lecithin, polyoxyethylene stearate, and polyethylene sorbitan monooleate; and preservatives such as ethyl, n-propyl, and p-hydroxybenzoate. Oily suspensions can be formulated by suspending the active ingredients in a vegetable oil, for example, arachis oil, olive oil, sesame oil or coconut oil, or in a mineral oil such as liquid paraffin. The oily suspensions can contain a thickening agent, for example beeswax, hard paraffin, or cetyl alcohol. These compositions can be preserved by the addition of an anti-oxidant such as ascorbic acid. Dispersible powders and granules (suitable for preparation of an aqueous suspension by the addition of water) can contain the active ingredients in admixture with a dispersing agent, wetting agent, suspending agent, or combinations thereof. Additional excipients can also be present.

The pharmaceutical compositions can also be in the form of oil-in-water emulsions. The oily phase can be a vegetable oil, for example olive oil or arachis oil, or a mineral oil, for example liquid paraffin or mixtures of these. Suitable emulsifying agents can be naturally-occurring gums, such as gum acacia or gum tragacanth; naturally-occurring phospholipids, such as soy lecithin; esters or partial esters derived from fatty acids and hexitol anhydrides, such as sorbitan monooleate; and condensation products of said partial esters with ethylene oxide, such as polyoxyethylene sorbitan monooleate.

Transdermal delivery can be accomplished by means of iontophoretic patches and the like. The active ingredients can also be administered in the form of suppositories for rectal administration of the drug. These compositions can be prepared by mixing the active agents with a suitable non-irritating excipient which is solid at ordinary temperatures but liquid at the rectal temperature and will therefore melt in the rectum to release the drug. Such materials include cocoa butter and polyethylene glycols.

Controlled release parenteral formulations of the compositions can be made as implants, oily injections, or as particulate systems. For a broad overview of delivery systems see, Banga, A. J., THERAPEUTIC PEPTIDES AND PROTEINS: FORMULATION, PROCESSING, AND DELIVERY SYSTEMS, Technomic Publishing Company, Inc., Lancaster, Pa., (1995) incorporated herein by reference. Particulate systems include microspheres, microparticles, microcapsules, nanocapsules, nanospheres, and nanoparticles.

Polymers can be used for ion-controlled release of active agents. Various degradable and nondegradable polymeric matrices for use in controlled drug delivery are known in the art (Langer R., Accounts Chem. Res., 26:537-542 (1993)). For example, the block copolymer, polaxamer 407 exists as a viscous yet mobile liquid at low temperatures but forms a semisolid gel at body temperature. It has shown to be an effective vehicle for formulation and sustained delivery of recombinant interleukin 2 and urease (Johnston et al., Pharm. Res., 9:425-434 (1992); and Pec et al., J. Parent. Sci. Tech., 44(2):58 65 (1990)). Alternatively, hydroxyapatite has been used as a microcarrier for controlled release of proteins (Ijntema et al., Int. J. Pharm., 112:215-224 (1994)). In yet another aspect, liposomes are used for controlled release as well as drug targeting of the lipid-capsulated drug (Betageri et al., LIPOSOME DRUG DELIVERY SYSTEMS, Technomic Publishing Co., Inc., Lancaster, Pa. (1993)). Numerous additional systems for controlled delivery of therapeutic proteins are known. See, e.g., U.S. Pat. Nos. 5,055,303, 5,188,837, 4,235,871, 4,501,728, 4,837,028 4,957,735 and 5,019,369, 5,055,303; 5,514,670; 5,413,797; 5,268,164; 5,004,697; 4,902,505; 5,506,206, 5,271,961; 5,254,342 and 5,534,496, each of which is incorporated herein by reference.

### V. Methods of Treating Cancer

Also provided herein are methods for the treatment of disorders such as cancer. The methods include administering to a therapeutically effective amount of a compound according to Formulas I, II, and/or III, optionally in combination with an antiandrogen drug.

In some embodiments, the disorder is a cancer. The cancer may be, for example, an androgen-independent cancer, a metastatic cancer, a castrate-resistant cancer, a castration recurrent cancer, a hormone-resistant cancer, a metastatic castrate-resistant cancer, or a combination thereof. In some embodiments, the cancer is prostate cancer or breast cancer.

In some embodiments, the method includes administration of an antiandrogen (e.g., enzalutamide, abiraterone, bicalutamide, darolutamide, and/or apalutamide). In some embodiments, the method includes administration of the antiandrogen and an STS inhibitor according to Formula I or Formula Ia as described above. In some embodiments, the method includes administration of the antiandrogen and an AR inhibitor according to Formula III or Formula IIIa as described above. In some embodiments, the method includes administration of a composition containing an antiandrogen and a compound according to Formula II or Formula IIa as described above. The active agents may be administered concomitantly or sequentially.

In some embodiments, the antiandrogen drug is a non-steroidal AR antagonist, a CYP17A1 inhibitor, or a combination thereof. Suitable non-steroidal AR antagonists include bicalutamide (Casodex, Cosudex, Calutide, Kalumid), flutamide, nilutamide, apalutamide (ARN-509, JNJ-56021927), darolutamide, enzalutamide (Xtandi), cimetidine and topilutamide. Suitable CYP17A1 inhibitors include abiraterone acetate (Zytiga), ketoconazole, and seviteronel. Any combination of antiandrogen drugs can be used in methods of the present invention.

The compounds and/or pharmaceutical compositions as described herein can be administered at any suitable dose in the methods. In general, the compound and/or composition is administered at a dose ranging from about 0.1 milligrams to about 1000 milligrams per kilogram of a subject's body weight (i.e., about 0.1-1000 mg/kg). In some embodiments, the compound and/or composition is administered at a dose ranging from about 1 milligram to about 100 milligrams per kilogram of a subject's body weight (i.e., about 1-100 mg/kg). The dose can be, for example, about 0.1-1000 mg/kg, or about 1-10 mg/kg, or about 10-50 mg/kg, or about 25-50 mg/kg, or about 50-75 mg/kg, or about 1-75-100 mg/kg, or about 1-500 mg/kg, or about 25-250 mg/kg, or about 50-100 mg/kg. The dose can be about 1, 2, 3, 4, 5, 10, 15, 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950 or 1000 mg/kg. The dosages can be varied depending upon the requirements of the patient, the severity of the disorder being treated, and the particular formulation being administered. The dose administered to a patient should be sufficient to result in a beneficial therapeutic response in the patient. The size of the dose will also be determined by the existence, nature, and extent of any adverse side-effects that accompany the administration of the drug in a particular patient. Determination of the proper dosage for a particular situation is within the skill of the typical practitioner. The total dosage can be divided and administered in portions over a period of time suitable to treat to the cancer or other disease/condition.

In some embodiments, the methods include administering the antiandrogen in an amount ranging from about 0.5 mg/kg/day to about 100 mg/kg/day (e.g., from about 1 mg/kg/day to about 10 mg/kg/day). As a non-limiting example, enzalutamide may be administered in an amount ranging from about 1 mg/kg/day to about 5 mg/kg day. In some embodiments, the methods include administering abiraterone or abiraterone acetate in an amount ranging from about 5 mg/kg/day to about 50 mg/kg/day.

The compounds and/or compositions can be administered for periods of time which will vary depending upon the nature of the particular disorder, its severity, and the overall condition of the subject to whom the compounds and/or compositions are administered. Administration can be conducted, for example, hourly, every 2 hours, three hours, four hours, six hours, eight hours, or twice daily including every 12 hours, or any intervening interval thereof. Administration can be conducted once daily, or once every 36 hours or 48 hours, or once every month or several months. Following treatment, a subject can be monitored for changes in his or her condition and for alleviation of the symptoms of the disorder. The dosage can either be increased in the event the subject does not respond significantly to a particular dosage level, or the dose can be decreased if an alleviation of the symptoms of the disorder is observed, or if the disorder has been remedied, or if unacceptable side effects are seen with a particular dosage. A therapeutically effective amount can be administered to the subject in a treatment regimen comprising intervals of at least 1 hour, or 6 hours, or 12 hours, or 24 hours, or 36 hours, or 48 hours between dosages. Administration can be conducted at intervals of at least 72, 96, 120, 144, 168, 192, 216, or 240 hours (i.e., 3, 4, 5, 6, 7, 8, 9, or 10 days).

In some embodiments, the methods further include administration of one or more additional anti-cancer agents. Examples of anti-cancer agents include, but are not limited to, chemotherapeutic agents (e.g., carboplatin, paclitaxel, pemetrexed, or the like), tyrosine kinase inhibitors (e.g., erlotinib, crizotinib, osimertinib, or the like), poly (ADP-ribose) polymerase inhibitors (e.g., olaparib, rucaparib, and the like), and immunotherapeutic agents (e.g., pembrolizumab, nivolumab, durvalumab, atezolizumab, or the like). In some embodiments, the methods include administration of radiotherapy, e.g., external beam radiation; intensity modulated radiation therapy (IMRT); brachytherapy (internal or implant radiation therapy); stereotactic body radiation therapy (SBRT)/stereotactic ablative radiotherapy (SABR); stereotactic radiosurgery (SRS); or a combination of such techniques.

In some of these embodiments, the cancer is advanced stage cancer. In some of these embodiments, the cancer is drug resistant. In some of these embodiments, the cancer is antiandrogen drug resistant or androgen independent. In some of these embodiments, the cancer is metastatic. In some of these embodiments, the cancer is metastatic and drug resistant (e.g., antiandrogen drug resistant). In some of these embodiments, the cancer is castration resistant. In some of these embodiments, the cancer is metastatic and castration resistant. In some of these embodiments, the cancer is enzalutamide resistant. In some of these embodiments, the cancer is enzalutamide and arbiraterone resistant. In some of these embodiments, the cancer is enzalutamide, arbiraterone, darolutamide, and bicalutamide resistant. In some of these embodiments, the cancer is enzalutamide, arbiraterone, bicalutamide, darolutamide, and apalutamide resistant. In other embodiments, the cancer is resistant (e.g., docetaxel, cabazitaxel, paclitaxel). The cancer (e.g., prostate or breast cancer) can be resistant to any combination of these drugs.

In some embodiments, treatment comprises inhibiting cancer cell (e.g., prostate or breast cancer cell) growth, inhibiting cancer cell proliferation, inhibiting cancer cell migration, inhibiting cancer cell invasion, ameliorating the symptoms of cancer, reducing the size of a cancer tumor, reducing the number of cancer tumors, reducing the number of cancer cells, inducing cancer cell necrosis, pyroptosis, oncosis, apoptosis, autophagy, or other cell death, or enhancing the therapeutic effects of a composition or pharmaceutical composition comprising a niclosamide analog and an antiandrogen drug. In particular instances, the subject does not have cancer.

In particular methods of treating cancer (e.g., prostate cancer, breast cancer, androgen-independent cancer, or drug-resistant cancer), described herein, treatment comprises enhancing the therapeutic effects of an antiandrogen drug (e.g., a non-steroidal adrogen receipt antagonist or a CYP17A1 inhibitor). In certain embodiments, treatment comprises enhancing the therapeutic effects of enzalutamide. In certain other embodiments, treatment comprises enhancing the therapeutic effects of abiraterone. In yet other embodiments, treatment comprises enhancing the therapeutic effects of apalutamide. In some other embodiments, treatment comprises enhancing the therapeutic effects of bicalutamide. The enhancement can be synergistic or additive.

In certain embodiments of the methods set forth herein, treatment comprises reversing, reducing, or decreasing cancer cell (e.g., prostate cancer cell or breast cancer cell) resistance to antiandrogen drugs. In certain embodiments of the methods set forth herein, treatment comprises resensitizing cancer cells (e.g., prostate cancer cells or breast cancer cells) to antiandrogen drugs. In any of the methods described herein, the antiandrogen drug is a compound selected from the group consisting of a non-steroidal androgen receptor antagonist, a CYP17A1 inhibitor, and a combination thereof. In certain embodiments, the antiandrogen drug is enzalutamide, apalutamide, bicalutamide, and/or abiraterone acetate.

In any of the aforementioned methods, treatment may comprise reversing cancer cell (e.g., prostate or breast cancer cell) resistance to an antiandrogen drug (e.g., a non-steroidal androgen receptor antagonist or CYP17A1 inhibitor); reducing or decreasing cancer cell resistance to an antiandrogen drug; or resensitizing cancer cells to an antiandrogen drug. In some embodiments, treatment comprises reversing cancer cell (e.g., prostate or breast cancer cell) resistance to enzalutamide, apalutamide, bicalutamide, darolutamide, abiraterone acetate, or a combination thereof. In some other embodiments, treatment comprises reducing or decreasing cancer cell resistance to enzalutamide, apalutamide, bicalutamide, darolutamide, abiraterone acetate, or a combination thereof. In some embodiments, treatment comprises resensitizing cancer cells to enzalutamide, apalutamide, bicalutamide, abiraterone acetate, darolutamide, or a combination thereof.

In any of the methods described herein, the cancer is selected from the group consisting of castration-resistant cancer, metastatic castration-resistant cancer, advanced stage cancer, drug-resistant cancer, antiandrogen-resistant cancer, bicalutamide resistant cancer, enzalutamide-resistant cancer, abiraterone acetate-resistant cancer, apalutamide-resistant cancer, darolutamide-resistant cancer, AR-V1-, AR-V3-, AR-V7-, AR-V9-, and/or AR-V12-induced drug-resistant cancer, AR-V1-, AR-V3-, AR-V7-, AR-V9-, and/or AR-V12-induced antiandrogen drug-resistant cancer, AR-V1-, AR-V3-, AR-V7-, AR-V9-, and/or AR-V12-induced enzalutamide-resistant cancer, AR-V1-, AR-V3-, AR-V7-, AR-V9-, and/or AR-V12-induced abiraterone acetate-resistant cancer, AR-V1-, AR-V3-, AR-V7-, AR-V9-, and/or AR-V12-induced apalutamide-resistant cancer, AR-V1-, AR-V3-, AR-V7-, AR-V9-, and/or AR-V12-induced bicalutamide-resistant cancer, and combinations thereof.

In some embodiments, a test sample is obtained from the subject. The test sample can be obtained before and/or after the STS inhibitor(s) and antiandrogen drug(s) are administered to the subject. Non-limiting examples of suitable samples include blood, serum, plasma, cerebrospinal fluid, tissue, saliva, and urine. In some instances, the sample comprises normal tissue. In other instances, the sample comprises cancer tissue. The sample can also be made up of a combination of normal and cancer cells.

In some embodiments, a reference sample is obtained. The reference sample can be obtained, for example, from the subject and can comprise normal tissue. The reference sample can be also be obtained from a different subject and/or a population of subjects. In some instances, the reference sample is either obtained from the subject, a different subject, or a population of subjects before and/or after the STS inhibitor(s) and antiandrogen drug(s) are administered to the subject, and comprises normal tissue. However, in some instances the reference sample comprises cancer tissue and is obtained from the subject and/or from a different subject or a population of subjects.

In some embodiments, the level of one or more biomarkers is determined in the test sample and/or reference sample. Non-limiting examples of suitable biomarkers include prostate-specific antigen (PSA), alpha-methylacyl-CoA racemase (AMACR), endoglin (CD105), engrailed 2 (EN-2), prostate-specific membrane antigen (PSMA), caveolin-1, interleukin-6 (IL-6), CD147, members of the S100 protein family (e.g., S100A2, S100A4, S100A8, S100A9, S100A11), annexin A3 (ANXA3), human kallikrein-2 (KLK2), TGF-Beta1, beta-microseminoprotein (MSMB), estrogen receptor (ER), progesterone receptor (PgR), HER2, Ki67, cyclin Dl, and cyclin E.

Prostate-specific antigen (PSA) is a protein produced primarily by prostate cells. Most PSA is released into the semen, but some PSA is also released into the blood. In the blood, PSA exists in unbound and complexed (cPSA) forms. Conventional laboratory tests can measure unbound and/or total (unbound and complexed) PSA. Elevated PSA levels can be caused by benign prostatic hyperplasia (BPH) and inflammation of the prostate, but can also be caused by prostate cancer. Determining PSA levels may also include one or more determinations of PSA velocity (i.e., the change in PSA level over time), PSA doubling time (i.e., how quickly the PSA level doubles), PSA density (i.e., a comparison of the PSA concentration and the volume of the prostate (which can be evaluated, for example, by ultrasound)), and age-specific PSA ranges.

Typically, the level of the one or more biomarkers in one or more test samples is compared to the level of the one or more biomarkers in one or more reference samples. Depending on the biomarker, and increase or a decrease relative to a normal control or reference sample can be indicative of the presence of cancer or a higher risk for cancer. As a non-limiting example, levels of one or biomarkers in test samples taken before and after the STS inhibitor(s) and antiandrogen drug(s) are administered to the subject are compared to the level of the one or more biomarkers in a reference sample that is either normal tissue obtained from the subject, or normal tissue that is obtained from a different subject or a population of subjects. In some instances, the biomarker is serum, and the level of PSA in a test sample obtained from the subject before the STS inhibitor(s) and antiandrogen drug(s) are administered to the subject is higher than the level of PSA in the reference sample. In other instances, the level of PSA in a test sample obtained from the subject after administration of the STS inhibitor(s) and antiandrogen drug(s) is decreased relative to the level of PSA in a test sample obtained prior to administration. Alternatively, as another non-limiting example, the difference in PSA level between a sample obtained from the subject after administration and a reference sample is smaller than a difference between the PSA level in a sample obtained from the subject prior to administration and the reference sample (i.e., administration results in a decrease in PSA in the test sample such that the difference between the level measured in the test sample and the level measured in the reference sample is diminished or eliminated).

The differences between the reference sample or value and the test sample need only be sufficient to be detected. In some embodiments, an increased level of a biomarker (e.g., PSA) in the test sample, and hence the presence of cancer or increased risk of cancer, is determined when the biomarker levels are at least, e.g., about 1-fold, 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, or 20-fold higher in comparison to a negative control. In other embodiments, a decreased level of a biomarker in the test sample, and hence the presence of cancer or increased risk of cancer, is determined when the biomarker levels are at least, e.g., about 1-fold, 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, or 20-fold lower in comparison to a negative control.

The biomarker levels can be detected using any method known in the art, including the use of antibodies specific for the biomarkers. Exemplary methods include, without limitation, PCR, Western Blot, dot blot, enzyme-linked immunosorbent assay (ELISA), radioimmunoassay (RIA), immunoprecipitation, immunofluorescence, FACS analysis, electrochemiluminescence, and multiplex bead assays (e.g., using Luminex or fluorescent microbeads). In some instances, nucleic acid sequencing is employed.

In certain embodiments, the presence of decreased or increased levels of one or more biomarkers is indicated by a detectable signal (e.g., a blot, fluorescence, chemiluminescence, color, radioactivity) in an immunoassay or PCR reaction (e.g., quantitative PCR). This detectable signal can be compared to the signal from a control sample or to a threshold value. In some embodiments, a decreased presence is detected, and the presence or increased risk of cancer is indicated, when the detectable signal of biomarker(s) in the test sample is at least, e.g., about 1-fold, 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, or 20-fold lower in comparison to the signal of antibodies in the reference sample or the predetermined threshold value. In other embodiments, an increased presence is detected, and the presence or increased risk of cancer is indicated, when the detectable signal of biomarker(s) in the test sample is at least, e.g., about 1-fold, 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, or 20-fold greater in comparison to the signal of antibodies in the reference sample or the predetermined threshold value.

### VI. Kits

Also provided herein are kits for preventing or treating cancer in a subject. The kits are useful for treating any cancer, some non-limiting examples of which include prostate cancer, breast cancer, uterine cancer, ovarian cancer, colorectal cancer, stomach cancer, pancreatic cancer, lung cancer (e.g., mesothelioma, lung adenocarcinoma), esophageal cancer, head and neck cancer, sarcomas, melanomas, thyroid carcinoma, CNS cancers (e.g., neuroblastoma, glioblastoma), chronic lymphocytic leukemia, and any other cancer described herein. The kits are also suitable for treating androgen-independent, castrate-resistant, castration recurrent, hormone-resistant, drug-resistant, and metastatic castrate-resistant cancers.

In some embodiments, the kits comprise a STS inhibitor and an antiandrogen drug. In some other embodiments, the kits further comprise a pharmaceutically acceptable carrier. In particular embodiments, the STS inhibitor is a compound according to Formulas I, II, and/or III.

In some embodiments, the antiandrogen drug is a non-steroidal androgen receptor antagonist, a CYP17A1 inhibitor, or a combination thereof. Suitable non-steroidal AR antagonists include bicalutamide (Casodex, Cosudex, Calutide, Kalumid), flutamide, nilutamide, apalutamide (ARN-509, JNJ-56021927), darolutamide, enzalutamide (Xtandi), cimetidine and topilutamide. Suitable CYP17A1 inhibitors include abiraterone acetate (Zytiga), ketoconazole, and seviteronel. Any combination of antiandrogen drugs can be used in the kits.

Materials and reagents to carry out the various methods described above can be provided in kits to facilitate execution of the methods. As used herein, the term “kit” includes a combination of articles that facilitates a process, assay, analysis, or manipulation. The kits may be utilized in a wide range of applications including, for example, diagnostics, prognostics, therapy, and the like.

Kits can contain chemical reagents as well as other components. In addition, the kits can include, without limitation, instructions to the kit user, apparatus and reagents for sample collection and/or purification, apparatus and reagents for product collection and/or purification, apparatus and reagents for administering STS inhibitor(s) and/or antiandrogen drug(s), apparatus and reagents for determining the level(s) of biomarker(s), sample tubes, holders, trays, racks, dishes, plates, solutions, buffers or other chemical reagents, suitable samples to be used for standardization, normalization, and/or control samples. Kits can also be packaged for convenient storage and safe shipping, for example, in a box having a lid.

In some embodiments, the kits also contain negative and positive control samples for detection of biomarkers. Non-limiting examples of suitable biomarkers include prostate-specific antigen (PSA), alpha-methylacyl-CoA racemase (AMACR), endoglin (CD105), engrailed 2 (EN-2), prostate-specific membrane antigen (PSMA), caveolin-1, interleukin-6 (IL-6), CD147, members of the S100 protein family (e.g., S100A2, S100A4, S100A8, S100A9, S100A11), annexin A3 (ANXA3), human kallikrein-2 (KLK2), TGF-Beta1, beta-microseminoprotein (MSMB), estrogen receptor (ER), progesterone receptor (PgR), HER2, Ki67, cyclin Dl, and cyclin E. In some instances, the one or more biomarkers comprises PSA. In some embodiments, the negative control samples are obtained from individuals or groups of individuals who do not have cancer. In other embodiments, the positive control samples are obtained from individuals or groups of individuals who have cancer. In some embodiments, the kits contain samples for the preparation of a titrated curve of one or more biomarkers in a sample, to assist in the evaluation of quantified levels of the one or more biomarkers in a test biological sample.

### VII. Examples

The present invention will be described in greater detail by way of specific examples. The following examples are offered for illustrative purposes only, and are not intended to limit the invention in any manner. Those of skill in the art will readily recognize a variety of noncritical parameters which can be changed or modified to yield essentially the same results.

### Example 1. Synthesis of STSi Compounds

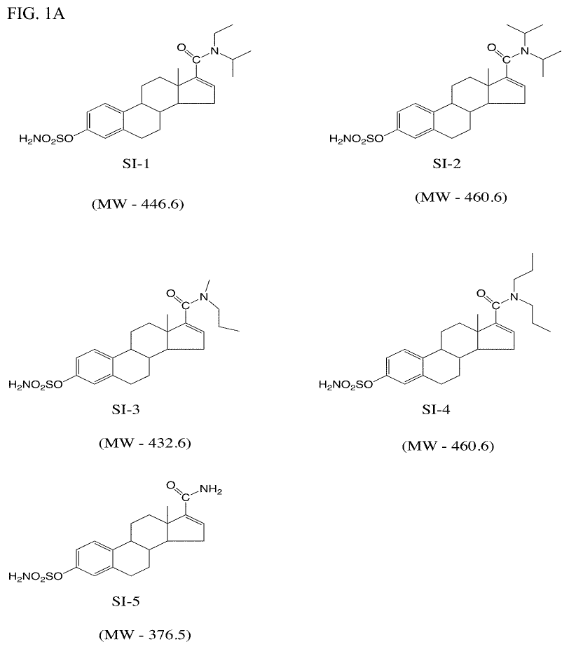

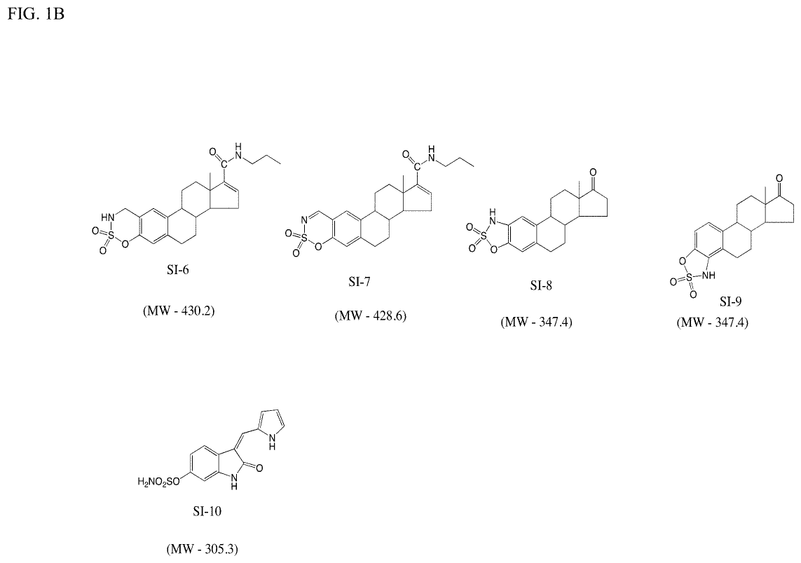

Synthesis of Si-1 and Si-2; A representative synthesis of STSi compounds Si-1 and Si-2 is shown below. Estrone was converted to the ditriflate (1). Select insertion of the substituted carboxamide into the D ring afforded compound (2). Removal of the triflate to yield (3) and sulfamoylation of (3) yielded Si-1. The synthesis of Si-2 was similar to Si-1 except diisopropyl amine was used instead of ethyl-isopropyl amine. A total of 10 STSi compounds (Si-1 to Si-10) were synthesized and are shown in FIG. 1A and FIG. 1B.

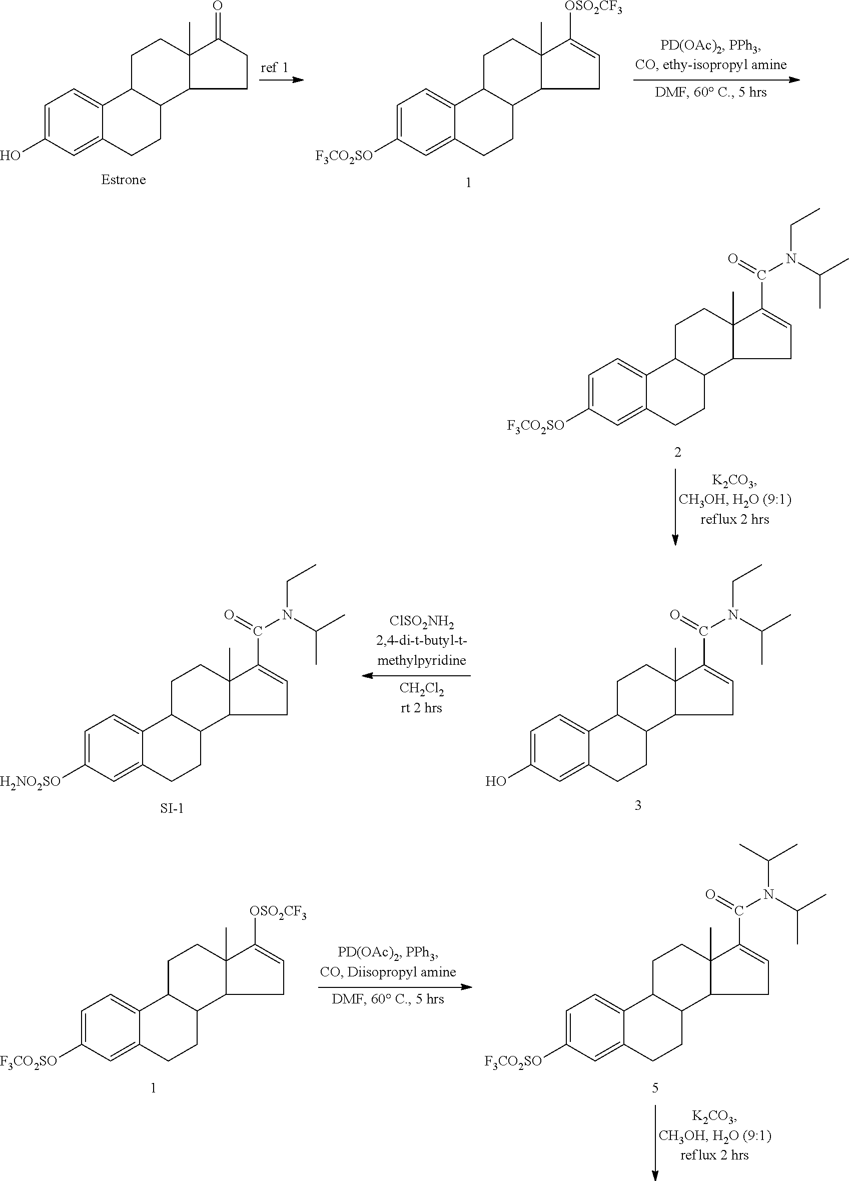

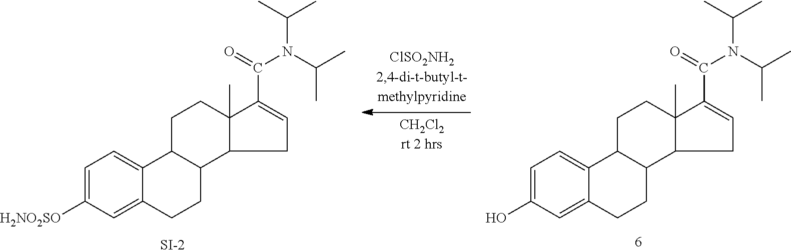

Synthesis of Si-8 and Si-9: The synthesis of STSi compound Si-8 is shown in the scheme below. Nitration of estrone provided a mixture of regioisomers (a) and (b). Reduction of compound (a) afforded amine (c). Protection of the amine with toysyl chloride yielded protected compound (d) with was further treated with sulfuryl chloride to obtain protected oxathiazolidine dioxide (e). Deprotection with aqueous base afforded the STSi compound Si-8. The analogous synthesis was performed using compound (b) to afford STSi compound Si-9. ¹H NMR (400 MHz, DMSO-d₆) δ 7.02 (d, J=4.6 Hz, 1H), 6.94 (d, J=4.7 Hz, 1H), 2.82 (dt, J=9.4, 4.5 Hz, 2H), 2.55-2.16 (m, 6H), 2.16-1.83 (m, 3H), 1.77 (dd, J=9.5, 3.8 Hz, 1H), 1.67-1.15 (m, 6H), 0.83 (d, J=4.6 Hz, 3H). Si-9: ¹H NMR (400 MHz, DMSO-d₆) δ 11.69 (s, 1H), 7.05 (q, J=8.6 Hz, 2H), 2.68 (dtd, J=24.0, 17.5, 6.2 Hz, 2H), 2.48-2.31 (m, 1H), 2.24 (td, J=10.5, 4.2 Hz, 1H), 2.16-1.86 (m, 3H), 1.86-1.68 (m, 1H), 1.68-1.08 (m, 5H), 0.83 (s, 3H).

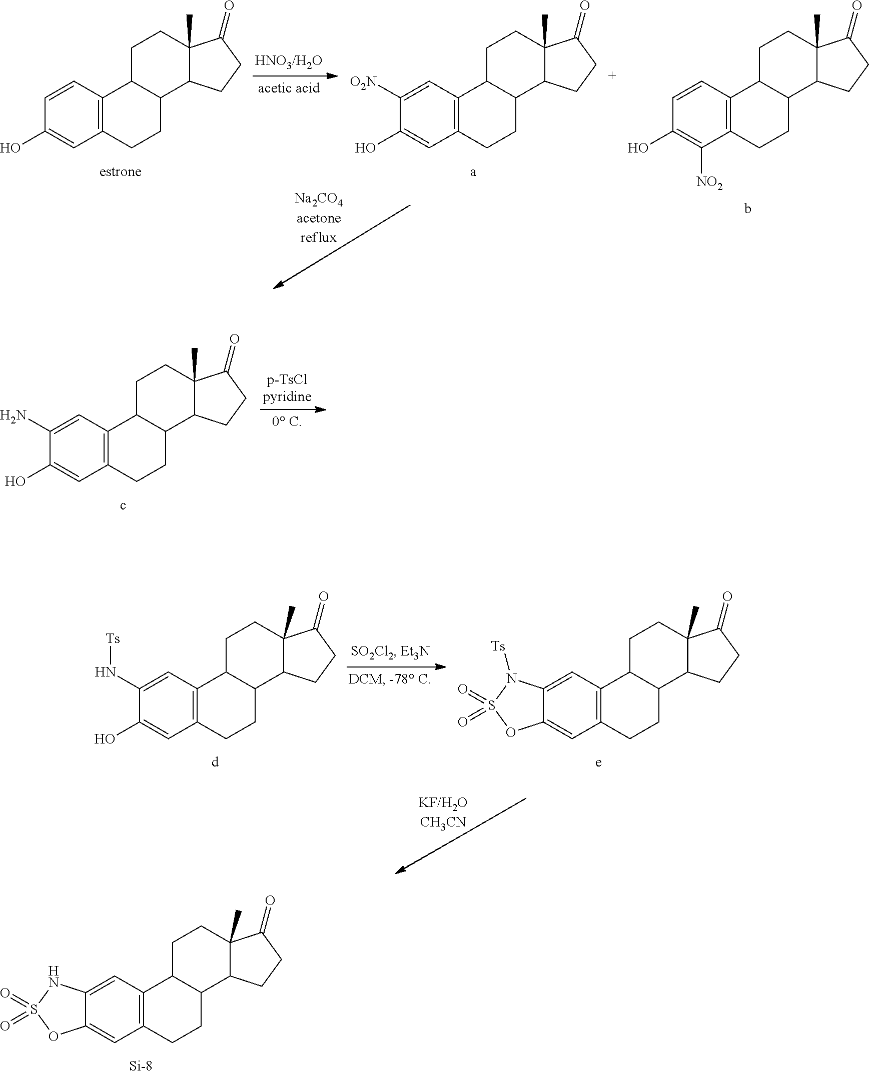

### Example 2. STSi Activity Study

The sensitivity of prostate cancer cells to STS inhibitors was tested using cell growth assays and clonogenic assays. Quantitative reverse transcription-PCR, and Western blotting were performed to detect expression levels of STS and AR. Expression of STS was downregulated using siRNA specific to STS. Steroid profile including DHEA and androgens was analyzed by Liquid Chromatography-Mass Spectrometry (LC-MS). STS activity was determined by 4-Methylumbelliferyl sulfate assay through a fluorescence microtiter plate reader. PSA secretion was determined by ELISA and PSA-luciferase activity was measured by reporter assay. Eleven potent STS inhibitors were synthesized and characterized. The in vivo efficacy of two novel STS inhibitors was tested in castration relapsed VCaP xenograft tumor models.

STS was found to be overexpressed in CRPC patients and cells. Inhibiting STS by siRNA was shown to suppress cell growth and AR signaling. Selected from 11 potential STS inhibitors, two novel small molecule inhibitors (Si-1 and Si-2) inhibited the STS activity and the growth of C4-2B and VCaP cells.

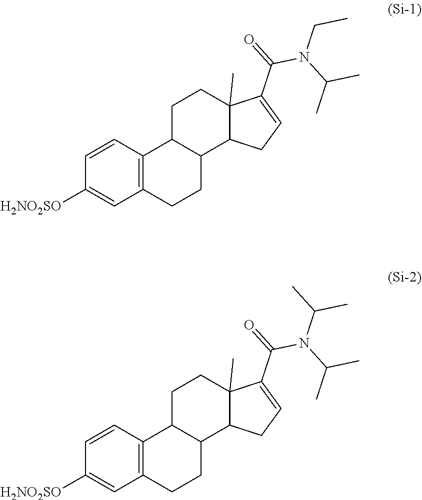

Additionally, Si-1 and Si-2 significantly suppressed AR expression and its transcriptional activity, suggesting that inhibition of STS activity by Si-1 and Si-2 downregulates the AR signaling. Both Si-1 and Si-2 significantly suppressed relapsed VCaP tumor growth and tumor AR levels in vivo. Furthermore, Si-1 increased the efficacy of enzalutamide treatment in vitro and in vivo.

### Example 3. Characterization of STSi in Prostate Cancer Cells

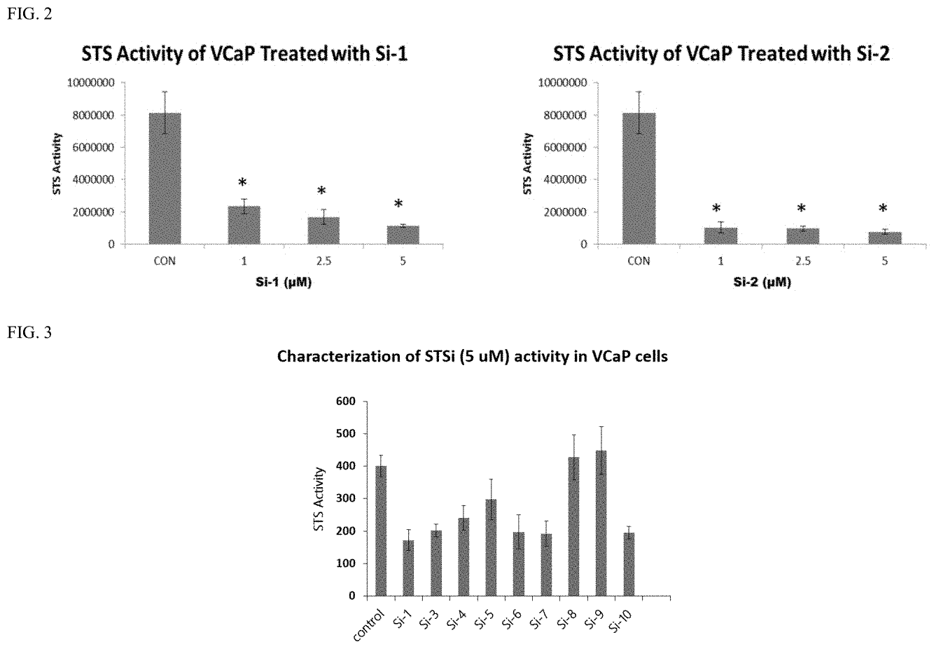

To evaluate the ability of STSi in inhibition of STS enzymatic activity, VCaP prostate cancer cells were treated with two synthesized STSi and STS enzymatic activity was measured. The activity assay was carried as described by Wolff, et al. (Anal Biochem, 2003. 318(2): p. 276-284). FIG. 2 shows that both STSi significantly inhibited STS enzymatic activity in a dose dependent manner. FIG. 3 shown the effects of other STSi on STS enzymatic activity in VCaP cells.

### Example 4. STSi Inhibits Prostate Cancer Cell Growth In Vitro

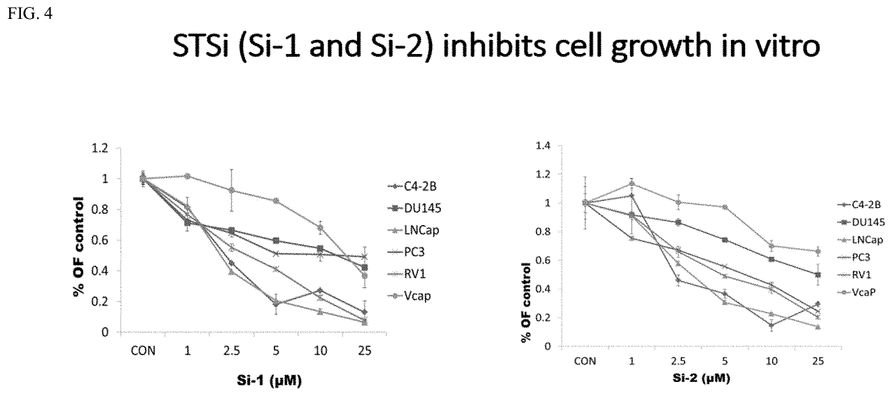

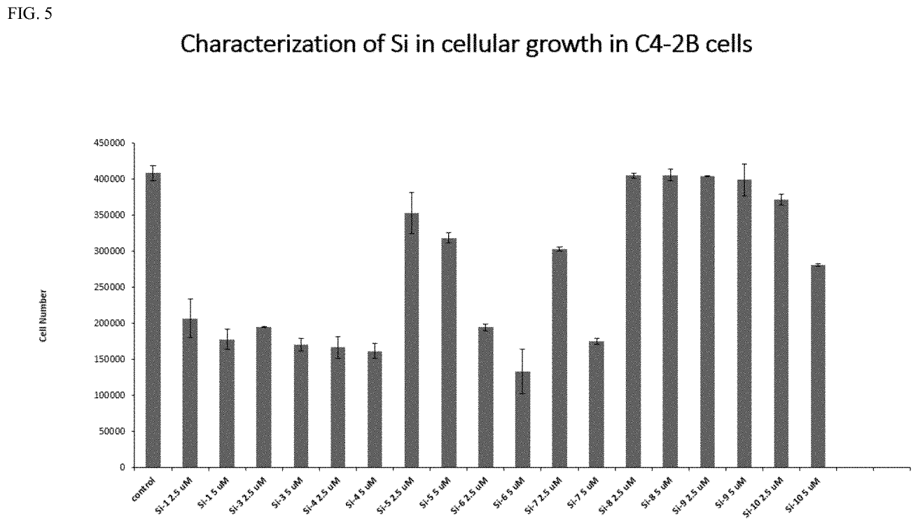

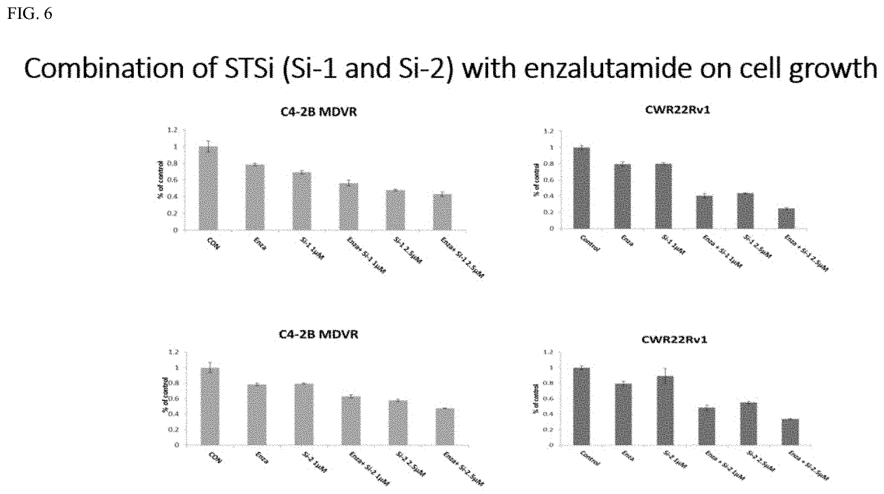

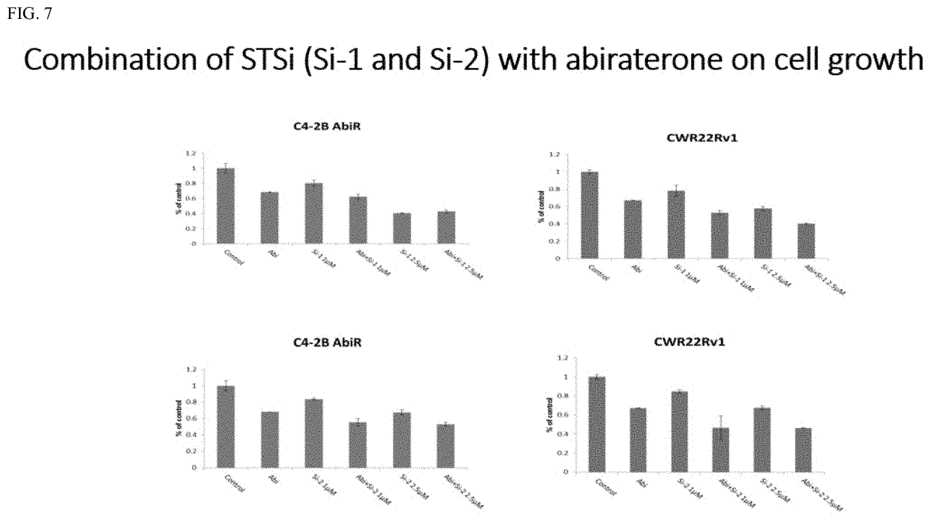

To examine the effects of STSi on the growth of prostate cancer cells, C4-2B, LNCaP, DU145, PC3, CWR22rv1 and VCaP cells were treated with increasing doses of STSi for 48 hrs and cell numbers were counted. As shown in FIG. 4, Si-1 and Si-2 inhibited cell growth in a dose dependent manner. FIG. 5 shows the effects of other STSi on the growth of C4-2B cells. To examine the effects of STSi on the growth of resistant prostate cancer cells, enzalutamide-resistant C4-2B MDVR and abiraterone-resistant C4-2BAbiR cells were treated with different doses of both STSi, and the cell numbers were counted. As shown in FIG. 6 and FIG. 7, the STSi's inhibit the growth of C4-2BMDVR and C4-2BAbiR cells, while combination with enzalutamide or abiraterone further decreased cell growth in both C4-2BMDVR and C4-2BAbiR cells.

### Example 5. STSi's Inhibit Prostate Cancer Tumor Growth In Vivo

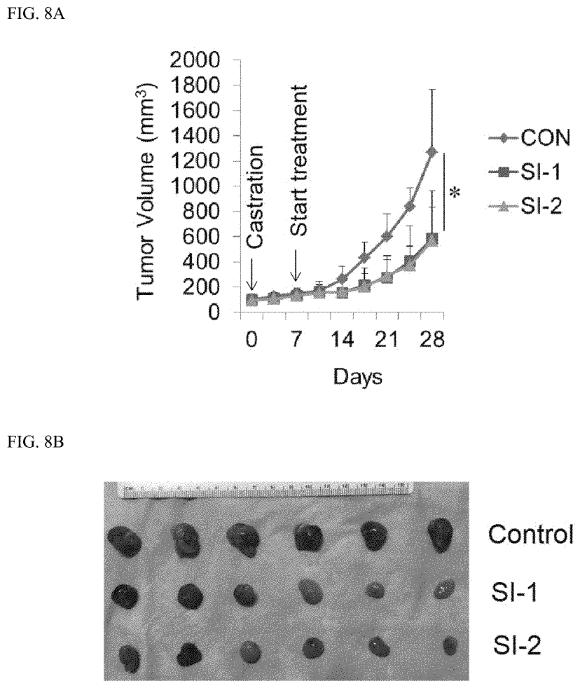

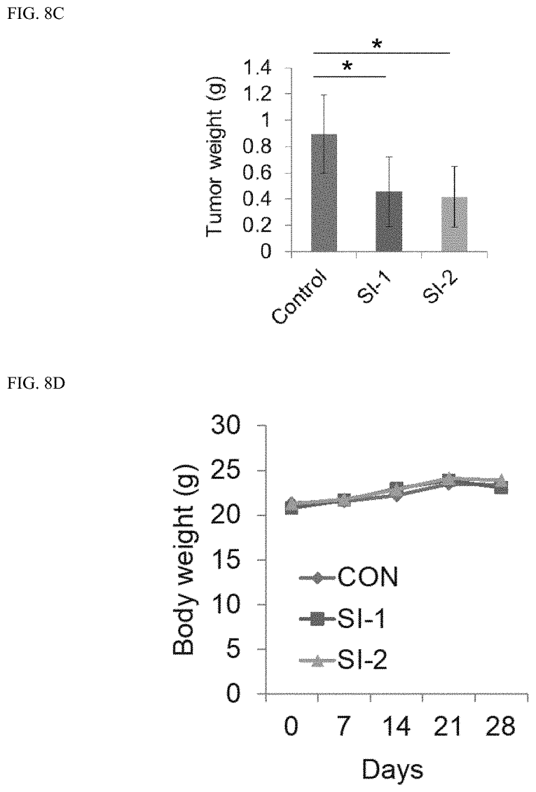

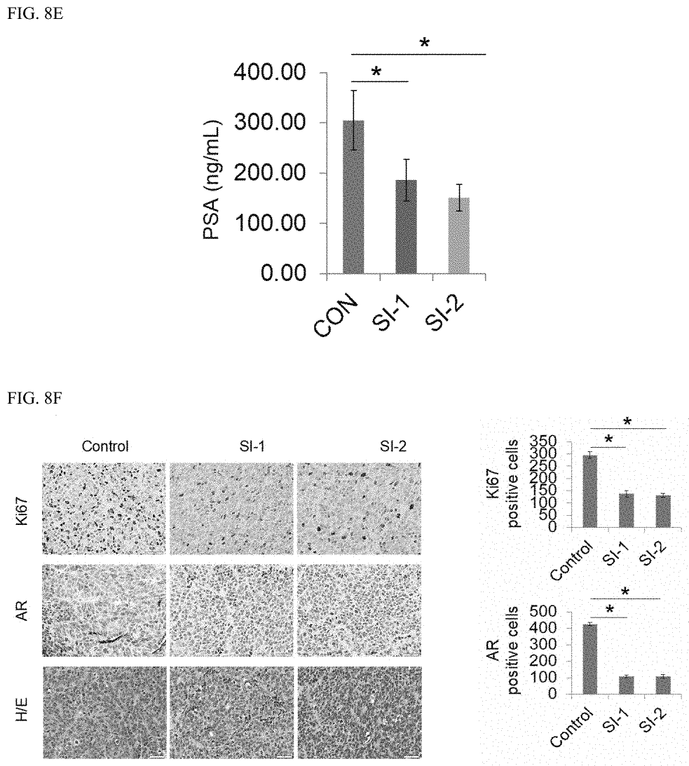

To determine whether these compounds inhibit enzalutamide/abiraterone resistance tumor growth in vivo, A VCaP tumor model that expresses endogenous STS was employed. VCaP tumors were allowed to develop in intact SCID mice and the animals were castrated after tumors reached 80-100 mm³ of size. This setting allowed VCaP, a CRPC line, to relapse after castration and STS inhibitor to be tested for their efficacy. As shown in FIG. 8A, after one week of castration, the VCaP tumors started to relapse and the treatments with Si-1 and Si-2 (25 mg/kg/d i.p.) significantly diminished tumor progression. Si-1 and Si-2 showed similar tumor suppression compared with control (around 60% tumor suppression) (FIG. 8B-8C). Both Si-1 and Si-2 significantly decreased the tumor weight after 3 weeks treatment (p=0.00155 and p=0.00061 respectively). However, the mice body weight was not affected by the Si-1 and Si-2 treatment compared with control group (FIG. 8D). The serum PSA level was also significantly decreased after 3 weeks of treatment. As shown in FIG. 8E, in a control group expressing 300 ng/mL PSA at 3 weeks of treatment time point, Si-1 decreased by 50% of PSA (p=0.00267) and Si-2 decreased by 55% of PSA (p=0.000692). Tumor proliferation and AR expression by IHC was also examined. As shown in FIG. 8E, both Si-1 and Si-2 showed less Ki67 and AR staining in VCaP tumors. The relapsed VCaP tumors expressed strong AR nuclear and cytoplasm staining. However, Si-1 and Si-2 significantly suppressed the tumor AR expression. Taken together, these in vivo results further confirm that Si-1 and Si-2 showed excellent anti-tumor efficacy in vivo.

### Example 6. STSi Improves Enzalutamide Treatment In Vitro and In Vivo

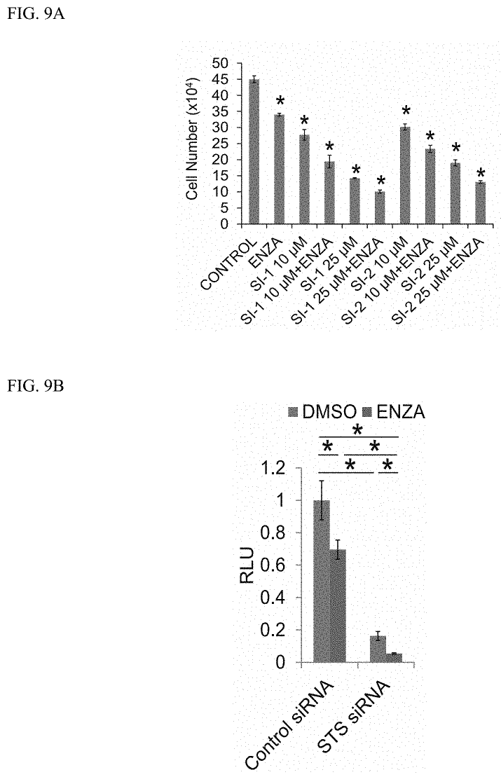

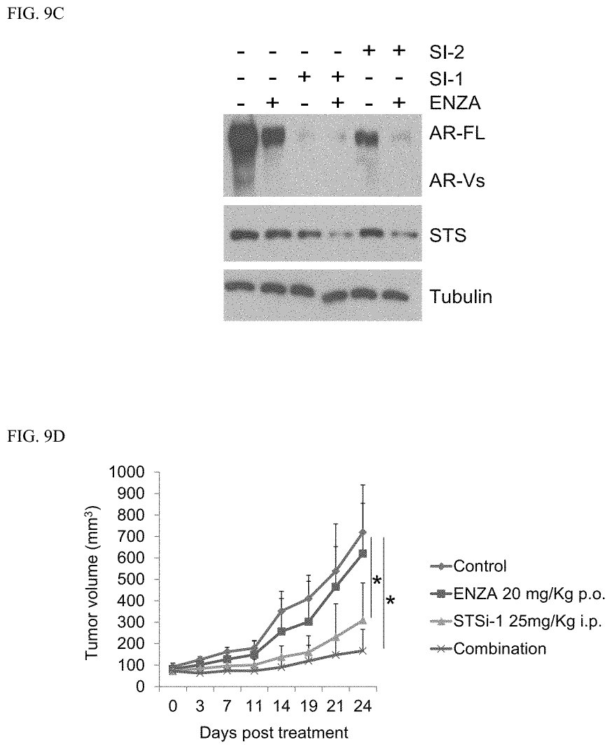

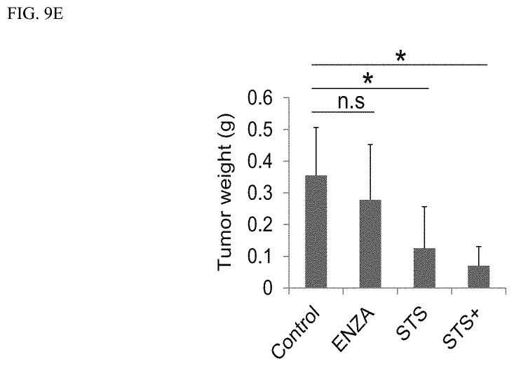

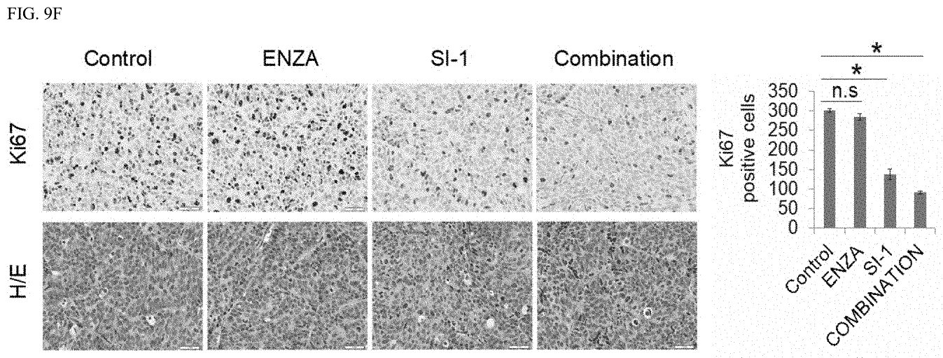

To further test if STS inhibition could improve enzalutamide treatment, Si-1 and Si-2 were tested in combination with enzalutamide in VCaP cells. As shown in FIG. 9A, enzalutamide slightly suppressed VCaP cell growth in vitro, and cell growth was reduced with addition of Si-1 and Si-2. Combination with Si-1 or Si-2 with enzalutamide further reduced cell numbers. The AR transcriptional activity was also determined, as shown in FIG. 9B. Enzalutamide treatment slightly decreased PSA luciferase activity in VCaP cells, STSi suppressed the AR activity, and in combination with enzalutamide further reduced the AR transcriptional activity. The AR expression was also determined by western blot. As shown in FIG. 9C, enzalutamide treatment decreased AR expression, both Si-1 and Si-2 significantly decreased AR expression, and combination treatment with Si-1 or Si-2 with enzalutamide completely suppressed AR expression.

The VCaP tumor model was then used to validate the efficacy of combination therapy. As shown in FIG. 9D-E, enzalutamide treatment alone only slighted delayed tumor growth and produced a tumor growth curve very similar to the control. Si-1 significantly suppressed tumor growth and tumor weight, and combined with enzalutamide further suppressed the tumor growth in vivo. IHC staining confirmed that enzalutamide did not affect the Ki67 expression in relapsed VCaP tumors, Si-1 significantly decreased the Ki67 expression, while combination treatment further lowered Ki67 expression (FIG. 9F). Taken together, these data demonstrate that Si-1 improved enzalutamide treatment in vivo.

### Example 7. STSi Inhibits Breast Cancer Cell Growth In Vitro

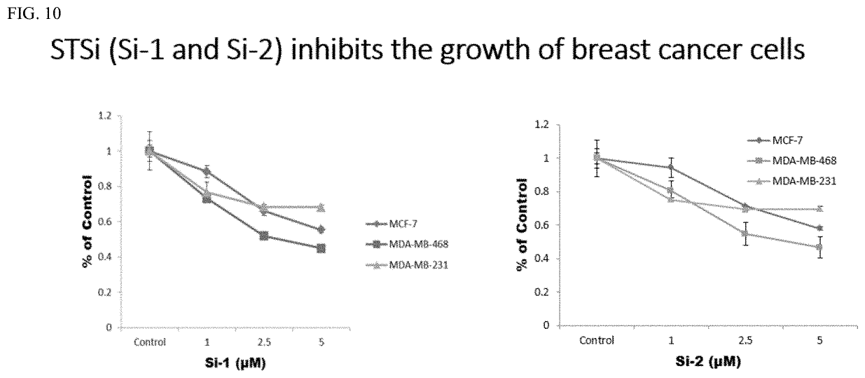

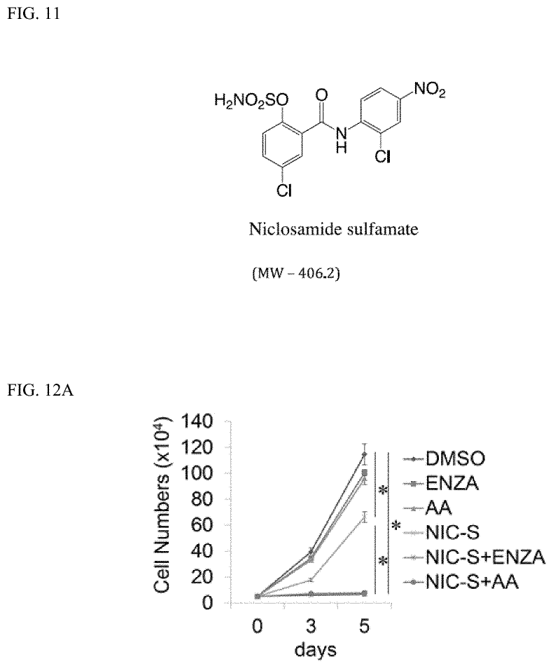

To examine the effects of STSi on the growth of breast cancer cells, MCF-7, MDA-MB-468 and MDA-MB-231 cells were treated with different doses of STSi for 48 hrs and cell numbers were counted. As shown in FIG. 10, Si-1 and Si-2 inhibits cell growth in a dose dependent manner.

### Example 8. Synthesis of Niclosamide Sulfamate

Niclosamide (3.27 g, 0.01 mol), triethylamine (TEA) (15.4 ml, 0.11 mol) and 4-(dimethylamino)pyridine (DMAP) (0.25 g) were dissolved in dichloromethane (DCM, CH₂Cl₂) (120 ml) at 0° C. Chlorosulfonamide (12.23 g, 0.105 mol) was then added to the reaction mixture and the resulting solution was stirred for 30 min at 0° C. The solution was then washed with water and the dichloromethane layer was obtained, dried and evaporated to yield a brownish solid. The solid was purified by silica column chromatography to obtain the white pure niclosamide sulfamate (0.64 g, 15.8% yield). ¹H NMR (300 MHz, Acetone-d₆) δ 8.81 (d, J=9.2 Hz, 1H), 8.40 (d, J=2.6 Hz, 1H), 8.32 (dd, J=9.2, 2.6 Hz, 1H), 8.01 (d, J=2.7 Hz, 1H), 7.75 (dd, J=8.8, 2.7 Hz, 1H), 7.65 (d, J=8.8 Hz, 1H).

### Example 9. Niclosamide Sulfamate Inhibits Wnt5A Signaling and Enhances Enzalutamide Treatment

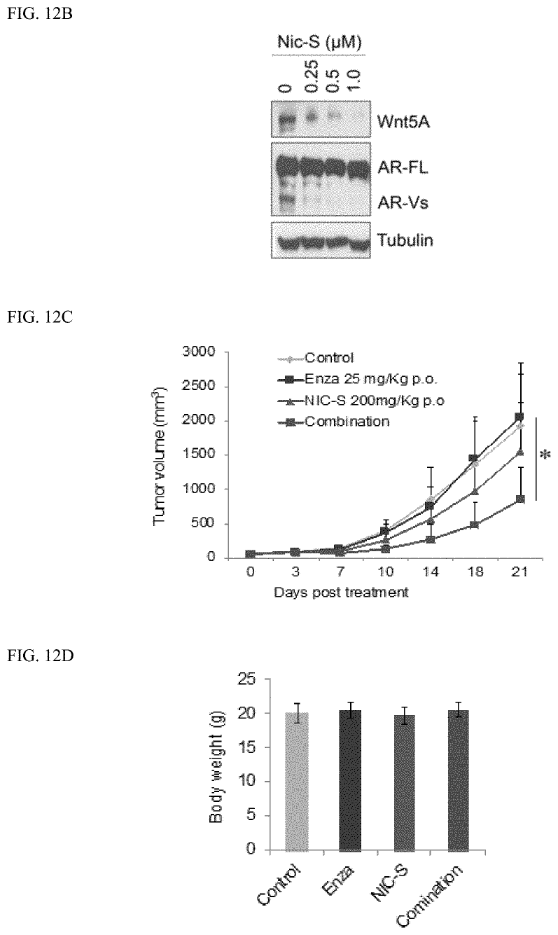

Niclosamide suffers from low solubility and oral bioavailability, which is believed to be due in part to the formation of intra-molecular hydrogen bonding between the phenolic OH group with the ketone group of niclosamide. The phenolic group was converted to a sulfamate to form the prodrug niclosamide sulfamate (Nic-S), which can be cleaved by steroid sulfatase after oral administration. Niclosamide sulfamate (Nic-S) was found to inhibit Wnt5A expression (FIG. 12B) and synergistically enhance enzalutamide treatment in vitro and in vivo without obvious toxicity (FIG. 12 A, C, D).

Although the foregoing has been described in some detail by way of illustration and example for purposes of clarity and understanding, one of skill in the art will appreciate that certain changes and modifications can be practiced within the scope of the appended claims. In addition, each reference provided herein is incorporated by reference in its entirety to the same extent as if each reference was individually incorporated by reference.

What is claimed is:

1. A compound according to Formula III: 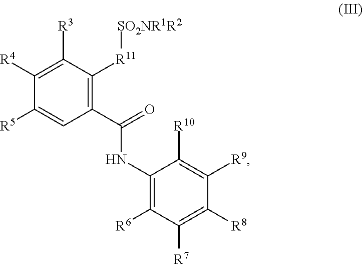
or pharmaceutically acceptable salt thereof, wherein:
R¹ and R² are each independently hydrogen or C₁₋₆ alkyl;
R³, R⁴, and R⁵ are each independently hydrogen, halogen, —OH, C₁₋₆ alkyl, or C₁₋₆ alkoxy;
R⁶, R⁷, R⁸, R⁹, and R¹⁰ are each independently hydrogen, halogen, —OH, —NH₃, —NO₂, —CN, C₁₋₆haloalkyl, C₁₋₆ alkyl, C₂₋₆ alkenyl, C₂₋₆ alkynyl, or C₁₋₆ alkoxy;
R¹¹ is a bond, C₁₋₆ alkylene, NR¹², or O; and
R¹² is hydrogen or C₁₋₆ alkyl.

2. The compound of claim 1, or a pharmaceutically acceptable salt thereof, having a structure according to Formula IIIa:

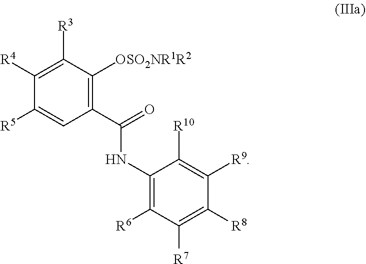

3. The compound of claim 2, or a pharmaceutically acceptable salt thereof, wherein
R¹ and R² are hydrogen; and
R³, R⁴, R⁵, R⁶, R⁷, R⁸, R⁹, and R¹⁰ are each independently hydrogen, halogen, or —NO₂.

4. The compound of claim 1, or a pharmaceutically acceptable salt thereof, wherein
R¹ and R² are hydrogen;
one of R³, R⁴, and R⁵ is halogen, and two of R³, R⁴, and R⁵ are hydrogen; and
one of R⁶, R⁷, R⁸, R⁹, and R¹⁰ is halogen, one of R⁶, R⁷, R⁸, R⁹, and R¹⁰ is —NO₂, and three of R⁶, R⁷, R⁸, R⁹, and R¹⁰ are hydrogen.

5. The compound of claim 1, or a pharmaceutically acceptable salt thereof, wherein
R¹ and R² are hydrogen;
one of R³, R⁴, and R⁵ is chloro, and the others of R³, R⁴, and R⁵ are hydrogen; and
one of R⁶, R⁷, R⁸, R⁹, and R¹⁰ is chloro, another of R⁶, R⁷, R⁸, R⁹, and R¹⁰ is —NO₂, and the others of R⁶, R⁷, R⁸, R⁹, and R¹⁰ are hydrogen.

6. The compound of claim 1, which is: 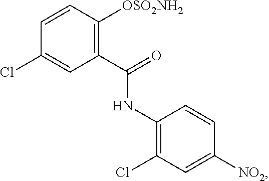
or a pharmaceutically acceptable salt thereof.

7. A pharmaceutical composition comprising the compound of claim 6, or a pharmaceutically acceptable salt thereof, and a pharmaceutically acceptable excipient.

8. The pharmaceutical composition of claim 7, further comprising an anti androgen drug.

9. A pharmaceutical composition comprising:
(i) an antiandrogen drug; and
(ii-a) a compound according to Formula I: 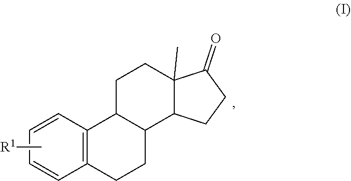
or a pharmaceutically acceptable salt thereof, wherein:
R¹ is —X(SO₂)Y—;
X is O and Y is NH, or X is NH and Y is O; and
R¹ is combined with two carbons of the phenyl group to which it is attached to form an oxathiazolidine dioxide;
or
(ii-b) a compound according to Formula II: 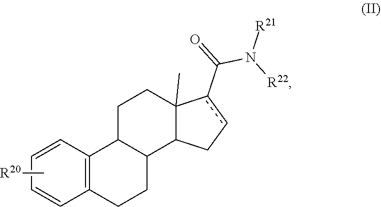
or a pharmaceutically acceptable salt thereof, wherein:
the dashed line represents a single bond or a double bond;
R²⁰ is —O(SO₂)NR²³R²⁴—, which is combined with two carbons of the phenyl group to which it is attached to form a 4- to 10-membered heterocycle, or
R²⁰ is —O(SO₂)NR²³R²⁵;
R²¹, R²², R²³, and R²⁵ are each independently hydrogen or C₁₋₆ alkyl; and
R²⁴ is a bond, C₁₋₆ alkylene, or C₁₋₆ alkenylene.

10. The pharmaceutical composition of claim 9, wherein the compound of Formula II has a structure according to Formula IIa:

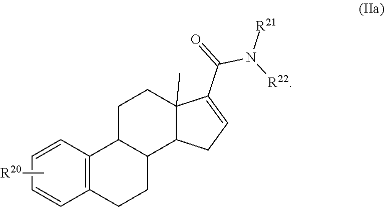

11. The pharmaceutical composition of claim 9, wherein R²⁰ is —O(SO₂)—NR²³R²⁵.

12. The pharmaceutical composition of claim 9, wherein:
R²¹ is hydrogen, methyl, ethyl, propyl, or isopropyl; and
R²² is hydrogen, propyl, or isopropyl.

13. The pharmaceutical composition of claim 9, wherein the compound of Formula I or the compound of Formula II is selected from the group consisting of:

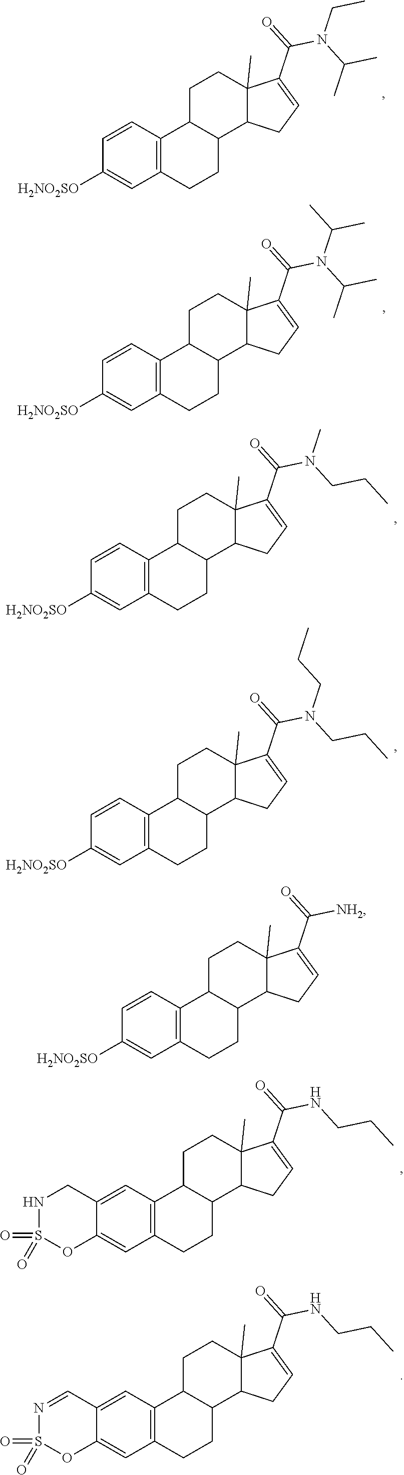

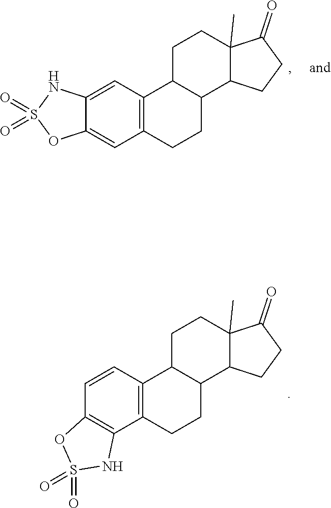 [SMILES: CC12CCC3c4cc5c(cc4CCC3C1CCC2=O)OS(=O)(=O)N5 ; CC12CCC3c4ccc5c(c4CCC3C1CCC2=O)NS(=O)(=O)O5]

14. The pharmaceutical composition of claim 9, wherein the antiandrogen drug is selected from the group consisting of enzalutamide, abiraterone, bicalutamide, apalutamide, darolutamide, and combinations thereof.

15. A method of treating cancer, the method comprising administering to a subject in need thereof a therapeutically effective amount of the compound claim 1.

16. The method of 15, wherein the cancer is selected from the group consisting of an androgen-independent cancer, a metastatic cancer, a castrate-resistant cancer, a castration recurrent cancer, a hormone-resistant cancer, a metastatic castrate-resistant cancer, and combinations thereof.

17. The method of claim 15, wherein the cancer is prostate cancer or breast cancer.

18. A method of treating cancer, the method comprising administering to a subject in need thereof a therapeutically effective amount of a pharmaceutical composition according to claim 9.

19. The method of claim 18, wherein the cancer is selected from the group consisting of an androgen-independent cancer, a metastatic cancer, a castrate-resistant cancer, a castration recurrent cancer, a hormone-resistant cancer, a metastatic castrate-resistant cancer, and combinations thereof.

20. The method of any one claim 19, wherein the antiandrogen drug is selected from the group consisting of enzalutamide, abiraterone, bicalutamide, apalutamide, darolutamide, and a combination thereof.
